(19) United States
(12) Patent Application Publication
(10) Pub. No.: US 2024/0390357 A1
(43) Pub. Date: Nov. 28, 2024

(54) PYRIDINES AND THEIR USE IN TREATMENT OF GBA-RELATED DISEASES

(71) Applicant: ZEVRA DENMARK A/S, Copenhagen N (DK)

(72) Inventors: Søren NEVE, Lyngby (DK); William Dalby BROWN, Søborg (DK); Kenneth THIRSTRUP, Søborg (DK)

(21) Appl. No.: 18/695,908

(22) PCT Filed: Sep. 27, 2022
(86) PCT No.: PCT/IB2022/059204

§ 371 Date: Mar. 27, 2024

(30) Foreign Application Priority Data
Sep. 28, 2021 (EP) 21199468.6

(51) Int. Cl. A61K 31/4545; A61K 31/437; A61K 31/506; A61P 25/16; C07D 213/38; C07D 213/64; C07D 239/26; C07D 241/12; C07D 471/04

(52) U.S. Cl. CPC A61K 31/4545; A61K 31/437; A61K 31/506; A61P 25/16; C07D 213/38; C07D 213/64; C07D 239/26; C07D 241/12; C07D 471/04

(57) ABSTRACT

The present invention relates to pyridines and their use for increasing GBA activity and/or levels as well as treatment of GBA-related diseases, such as Parkinson's disease.

### TECHNICAL FIELD

The present invention relates to pyridines, and their use for increasing GBA activity and/or levels as well as treatment of GBA-related diseases, such as Parkinson's disease.

### BACKGROUND

The lysosome functions as a crucial re-processing center in human cells, breaking down proteins and fatty substances, such as glycosphingolipids, into their basic building blocks that are then recycled. A set of rare genetic diseases, called lysosomal storage diseases (LSD), are the result of carrying a distinct mutation in both copies of certain genes which encode various lysosomal enzymes. Gaucher disease, the most common lysosomal storage disease, is the result of a mutation in both copies of the GBA1 gene that codes for the Glucocerebrosidase (GCase) enzyme. Such homozygous mutations in both copies of the GBA1 gene cause a severe loss of up to 95% of GCase activity. As a result of this critical loss of enzyme activity, the metabolism of certain glycosphingolipids is significantly impaired in Gaucher disease patients, leading to accumulation of Glucosylceramide (GluCer), the GCase enzyme's substrate. This accumulation leads to serious health issues and organ pathology.

Many of these GBA mutations are also found in patients with Parkinson's disease (PD). Heterozygous mutations as found in GBA mutation carriers (having one mutated GBA gene) are found to predispose for development of Parkinson's disease (Gan-Or et al., Neurology, 2015). Mutations in GBA are now considered one of the main genetic risk factors for Parkinson's disease. It has been estimated that at least 8% of patients with Parkinson's disease have mutations in the GBA gene, both mild and severe GBA mutations, including L444P heterozygotes. Also secondary deficiencies of GBA activity may be linked to Parkinson's disease.

State of the art compounds, Ambroxol and LTI-291 have been shown to increase GBA activity, an important effect in treatment of GBA-mediated disorders. In order to meet the medical need of treating GBA-mediated disorders, more and better compounds are needed.

### SUMMARY

The present inventors have developed a series of compounds that effectively act as GBA inducers with completely different structural chemotype compared to state of the art compounds Ambroxol and LTI-291. This renders the compounds of the present disclosure promising candidates for treatment of GBA-mediated disorders

In a first aspect, a compound of formula (I) is provided,

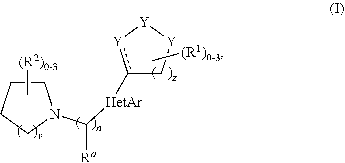

or a pharmaceutically acceptable salt or solvate thereof; wherein
HetAr is a monocyclic or bicyclic heteroaryl comprising from 1 to 4 nitrogen atoms and from 0 to 2 oxygen atoms in the ring with the remainder of ring-atoms being carbon;
wherein HetAr is optionally substituted;
each Y is independently selected from the group: N, NH, CH, and CH₂;
z is 1 or 2;
n is 1 or 2;
v is 1 or 2;
each R^{a} is independently selected from the group consisting of: hydrogen, alkyl, halogen, hydroxy, alkoxy, amino, amido, acyl, cycloalkyl, and heterocycloalkyl;
each R¹ is independently selected from the group consisting of: hydrogen, alkyl, halogen, hydroxy, alkoxy, amino, amido, acyl, cycloalkyl, and heterocycloalkyl;
each R² is independently selected from the group consisting of: hydrogen, alkyl, halogen, hydroxy, alkoxy, amino, amido, acyl, cycloalkyl, and heterocycloalkyl; and
“ [symbol] ” is a single bond or a double bond.

In a second aspect, a compound is provided of formula (IIa) or formula (IIb),

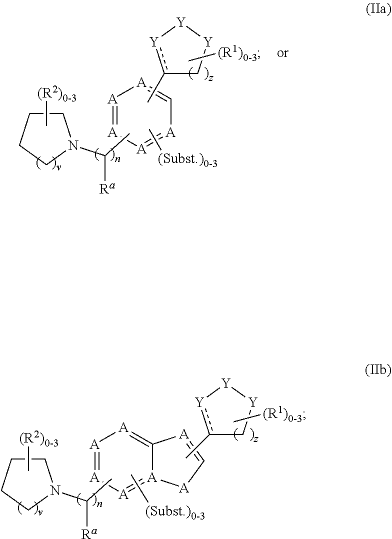

wherein each A is independently selected from the group: N, NH, C, CH, and CH₂; and
wherein each Subst is independently selected from the group consisting of hydrogen, halogen, hydroxyl, alkoxy, amino, amido, alkyl, and cycloalkyl.

In a third aspect, a compound of formula (IIIa) or formula (IIIb) is provided,

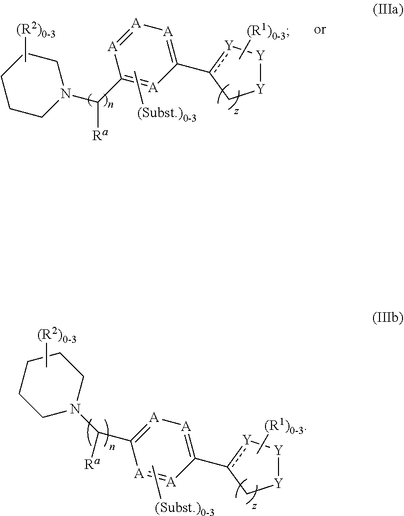

In a fourth aspect, a pharmaceutical composition is provided comprising a compound as defined herein, and one or more pharmaceutically acceptable adjuvants, excipients, carriers, buffers and/or diluents.

In a fifth aspect, a method for treating a disease in a subject is provided comprising administering a compound as defined herein to the subject, wherein the disease is associated with reduced GBA levels and/or activity.

In a sixth aspect, a method of increasing the GBA activity and/or levels is provided comprising contacting GBA with a compound as defined herein.

In a seventh aspect, a compound as defined herein is provided for the manufacture of a medicament for the treatment of Parkinson's disease (PD).

### DETAILED DESCRIPTION

### Definitions

With reference to substituents, the term “independently” refers to the situation where when more than one substituent is possible, the substituents may be the same or different from each other.

As used herein, the term “pharmaceutically acceptable salt” refers to a salt used typically in the pharmaceutical field. Examples of the pharmaceutically acceptable salt include sodium salts, hydrochloride salts, magnesium salts, calcium salts, trifluoroacetic acid salts and potassium salts, but are not limited thereto. Further exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, olcate, tannate, pantothenate, bitartrate, ascorbate, succinate, malcate, gentisinate, fumarate, gluconate, glucuronate, saccharate, formate, benzoate, glutamate, methanesulfonate, ethanesulfonate, benzenesulfonate, p-toluenesulfonate, and pamoate.

The potency, “EC_{1.5}” referred to herein is determined based on the dose response effects of the compounds as the concentration where “Percent GCase activity”=150% corresponding to at 1.5-fold induction of GCase activity.

The term “alkyl” refers to a straight or branched hydrocarbon radical consisting of carbon and hydrogen atoms, and may be straight or branched, substituted or unsubstituted. The alkyl may be cyclic or non-cyclic. In some preferred embodiments, the alkyl group may consist of 1 to 12 carbon atoms, e.g. 1 carbon atom, 2 carbon atoms, 3 carbon atoms, 4 carbon atoms etc., up to and including 12 carbon atoms. In one embodiment, a C₁₋₆ alkyl is provided, which comprises 1-6 carbon atoms.

Exemplary alkyl groups include, but are not limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of any suitable substituents. An alkyl group can be mono-, di-, tri- or tetra-valent, as appropriate to satisfy valence requirements.

Generally, suitable substituents for substituted groups disclosed herein independently include, but are not limited to, alkyl, heteroalkyl, alkenyl, alkynyl, cycloalkyl, heterocycloalkyl, aryl, arylalkyl, heteroaryl, heteroarylalkyl, hydroxy, halo, hydroxyl, cyano, trifluoromethyl, trifluoromethoxy, nitro, trimethylsilanyl, —OR^{b}, —SR^{b}, —OC(O)—R^{b}, —N(R^{b})₂, —C(O)R^{b}, —C(O)OR^{b}, —OC(O)N(R^{b})₂, —C(O)N(R^{b})₂, —N(R^{b})C(O)OR^{b}, —N(R^{b})C(O)R^{b}, —N(R^{b})C(O)N(R)₂, N(R^{b})C(NR^{b})N(R^{b})₂, —N(R^{b})S(O)ₜR^{b}, —N(R^{b})S(O)₂R^{b}, —S(O)OR^{b}, —S(O)₂OR^{b}, —S(O)N(R^{b})₂, —S(O)₂N(R^{b})₂, or PO₃(R^{b})₂ where each R^{b} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl.

The term “cycloalkyl” refers to a monocyclic or polycyclic radical that contains carbon and hydrogen, and may be saturated, or partially unsaturated. In some preferred embodiments, cycloalkyl groups include groups having from 3 to 12 ring atoms (i.e. (C₃₋₁₂) cycloalkyl or C(₃₋₁₂)cycloalkyl). Whenever it appears herein, a numerical range such as “3 to 12” in (C₃₋₁₂)cycloalkyl or C(₃₋₁₂)cycloalkyl refers to each integer in the given range—e.g., “3 to 12 carbon atoms” means that the cycloalkyl group may consist of 3 carbon atoms, 4 carbon atoms, 5 carbon atoms, etc., up to and including 12 carbon atoms. Illustrative examples of cycloalkyl groups include, but are not limited to cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

The term “alkoxy” refers to the group —O-alkyl. In some preferred embodiments, the alkoxy group contains from 1 to 12 carbon atoms of a straight, branched, cyclic configuration and combinations thereof attached to the parent structure through an oxygen. Examples of alkoxy include, but are not limited to, methoxy, ethoxy, propoxy, isopropoxy, cyclopropyloxy and cyclohexyloxy.

The term “acyl” refers to R^{c}—(C═O)— wherein R^{c} include, but is not limited to, alkyl, heteroalkyl, alkenyl, alkynyl, cycloalkyl, heterocycloalkyl, aryl, aralkyl, heteroaryl. The acyl is attached to the parent structure through the carbonyl functionality.

The term “amino” or “amine” refers to a —N(R^{b})₂ radical group, where each R^{b} is independently hydrogen, alkyl, (halo)alkyl, alkenyl, alkynyl, carbocyclyl, carbocyclylalkyl, aryl, aralkyl, heterocycloalkyl, heterocycloalkylalkyl, heteroaryl or heteroarylalkyl, unless stated otherwise. When a —N(R^{b})₂ group has two R^{b} substituents other than hydrogen, they can be combined with the nitrogen atom to form a 4-, 5-, 6- or 7-membered ring. For example, —N(R^{b})₂ is intended to include, but is not limited to, 1-pyrrolidinyl, 1-piperazinyl, and 4-morpholinyl.

The term “amide” or “amido” refers to a chemical moiety with formula —(C═O)N(R^{d})₂ or —NH(C═O)R^{d}, where R^{d} is selected from the group consisting of hydrogen, alkyl, alkenyl, alkynyl, carbocyclyl, carbocyclylalkyl, cycloalkyl, aryl, and heteroaryl. The R^{d} of —N(R^{d})₂ of the amide may optionally be taken together with the nitrogen to which it is attached to form a 4-, 5-, 6- or 7-membered ring. Unless stated otherwise specifically in the specification, an amide group is optionally substituted independently by one or more of the substituents as described herein as suitable substitution groups.

The term “haloalkyl” refers to an alkyl radical, as defined above, that is substituted by one or more halogen atoms. The term “alkyl” thus includes “haloalkyl”. Examples of haloalkyl include, but are not limited to, trifluoromethyl, difluoromethyl, 2,2,2-trifluoroethyl, 1-fluoromethyl-2-fluoroethyl, and the like.

The term “halo”, “halide”, or, alternatively, “halogen” is intended to mean fluoro, chloro, bromo or iodo.

The term “aromatic” means an unsaturated, cyclic and planar hydrocarbon group with a delocalized conjugated π system having 4n+2π electrons, where n is an integer having a value of 0, 1, 2, 3, and so on. In some embodiments, the aromatic group is an “aryl” (abbreviated as Ar), which refers to an aromatic radical with six to ten ring atoms (e.g., (C₆₋₁₀)aromatic or (C₆₋₁₀)aryl) which has at least one ring having a conjugated pi electron system which is carbocyclic (e.g., phenyl, fluorenyl, and naphthyl).

The term “aralkyl” or “arylalkyl” refers to an (aryl)alkyl-radical where aryl and alkyl are as disclosed herein.

The term “heteroaryl” or “heteroaromatic refers to a 5- to 18-membered aromatic radical (e.g., (C₅₋₁₃)heteroaryl) that includes one or more ring heteroatoms selected from nitrogen, oxygen and sulfur, and which may be a monocyclic, bicyclic, tricyclic or tetracyclic ring system. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Heteroaryl groups may be substituted.

### Compounds

In one embodiment, a compound of formula (I) is provided,

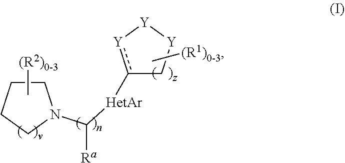

or a pharmaceutically acceptable salt or solvate thereof; wherein
HetAr is a monocyclic or bicyclic heteroaryl comprising from 1 to 4 nitrogen atoms and from 0 to 2 oxygen atoms in the ring with the remainder of ring-atoms being carbon,
wherein HetAr is optionally substituted;
each Y is independently selected from the group: N, NH, CH, and CH₂;
z is 1 or 2;
n is 1 or 2;
v is 1 or 2;
each R^{a} is independently selected from the group consisting of: hydrogen, alkyl, halogen, hydroxy, alkoxy, amino, amido, acyl, cycloalkyl, and heterocycloalkyl;
each R¹ is independently selected from the group consisting of: hydrogen, alkyl, halogen, hydroxy, alkoxy, amino, amido, acyl, cycloalkyl, and heterocycloalkyl;
each R² is independently selected from the group consisting of: hydrogen, alkyl, halogen, hydroxy, alkoxy, amino, amido, acyl, cycloalkyl, and heterocycloalkyl; and
“ [symbol] ” is a single bond or a double bond.

In one embodiment, the compound is provided wherein HetAr is a monocyclic heteroaryl. In one embodiment, HetAr is a monocyclic heteroaryl comprising 1, 2, 3, or 4 nitrogen atoms in the ring, for example 1 or 2 nitrogen atoms. In one embodiment, HetAr is a monocyclic heteroaryl comprising 0, 1, or 2 oxygen atoms in the ring. In one embodiment, HetAr is a bicyclic heteroaryl. In one embodiment, HetAr is a bicyclic heteroaryl comprising 1, 2 or 3 nitrogen atoms in the ring. In one embodiment, HetAr is a bicyclic heteroaryl comprising 0, 1, or 2 oxygen atoms in the ring. In one embodiment, HetAr comprises 5-10 ring atoms.

In one embodiment, the compound is of formula (IIa) or formula (IIb),

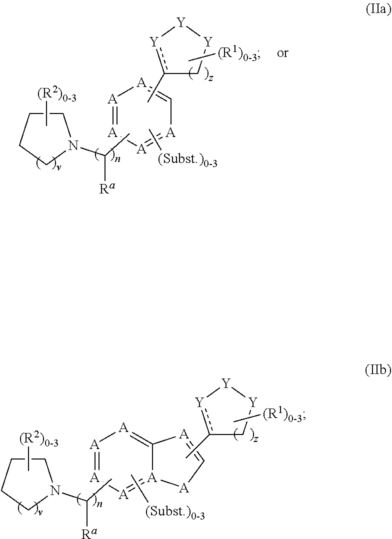

wherein each A is independently selected from the group: N, NH, C, CH, and CH₂; and
wherein each Subst is independently selected from the group consisting of hydrogen, halogen, hydroxyl, alkoxy, amino, amido, alkyl, and cycloalkyl.

In one embodiment, the compound is of formula (IIIa) or formula (IIIb),

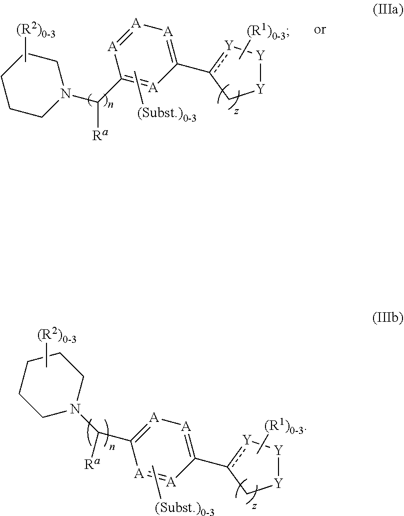

In one embodiment, the bond denoted “ [symbol] ” is a single bond. In another embodiment, the bond denoted “ [symbol] ” is a double bond.

In one embodiment, the term “n” of any of the formulas presented herein is 1.

In one embodiment, R^{a} is hydrogen, C₁₋₆ alkyl, or halogen. In one embodiment, R^{a} is hydrogen or methyl. In one embodiment, “n” of any of the formulas presented herein is 1 and wherein R^{a} is hydrogen, C₁₋₆ alkyl, or halogen. In one embodiment, the compound is provided wherein “n” is 1 and wherein R^{a} is hydrogen or methyl.

In one embodiment, a compound is provided according to any of the formulas described herein wherein “v” is 2. This provides a six membered ring.

In one embodiment, a compound is provided according to any of the formulas described herein wherein one R¹ is hydrogen or C₁₋₆ alkyl.

In one embodiment, a compound is provided according to any of the formulas described herein wherein one Y is NH and each other Y is independently CH or CH₂. In one embodiment, one Y is N which is bound to one R₁; and wherein each other Y is independently CH or CH₂. In one embodiment, one Y is N which is bound to one R₁ which is hydrogen or C₁₋₆ alkyl; and wherein each other Y is independently CH or CH₂.

In one embodiment, the compound is provided wherein R₂ is absent.

In one embodiment, the compound is provided comprising one or two R₂ independently selected from the group consisting of: halogen and alkyl. In one embodiment, the compound comprises one R₂ which is a C₁₋₆ alkyl.

In one embodiment, the compound comprises one R₂ which is halogen, such as fluorine.

In one embodiment, the compound is provided according to formulas (IIa), (IIb), (IIIa) or (IIIb) wherein each Subst is independently selected from the group consisting of: hydrogen, C₁₋₆ alkyl, and C₁₋₆ alkoxy. In one embodiment, the compound comprises one Subst selected from the group consisting of: hydrogen, C₁₋₆ alkyl, and C₁₋₆ alkoxy.

In one embodiment, the compound is provided according to formulas (IIa), (IIb), (IIIa) or (IIIb) wherein two A are each N or NH, and the remainder of A are each C, CH or CH₂. In one embodiment, one A is N or NH, and the remainder of A are each C, CH or CH₂.

In one embodiment, the compound is of formula (IIb) and one A is O, and one A is N or NH, and the remainder of A are each C, CH or CH₂.

In a particular embodiment, the compound is selected from the group consisting of:

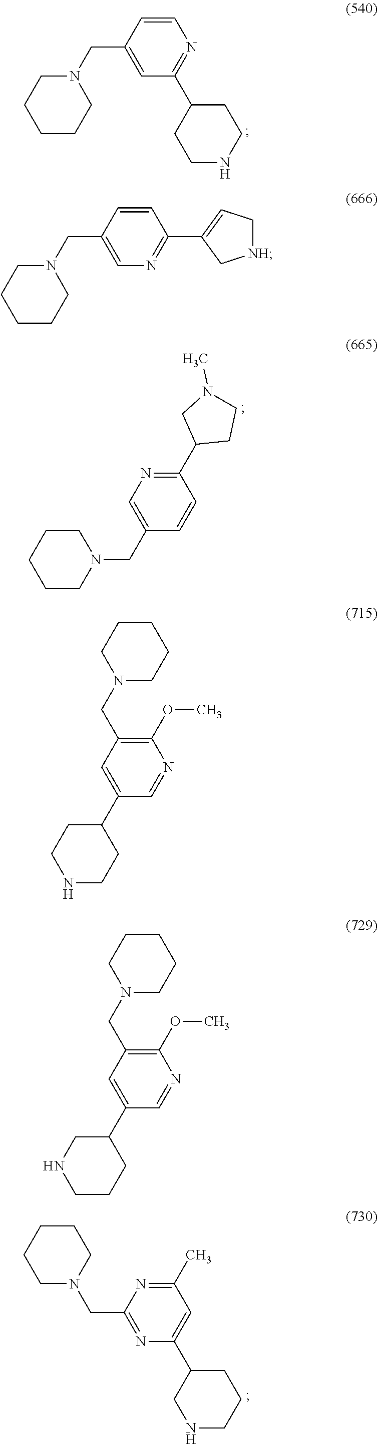

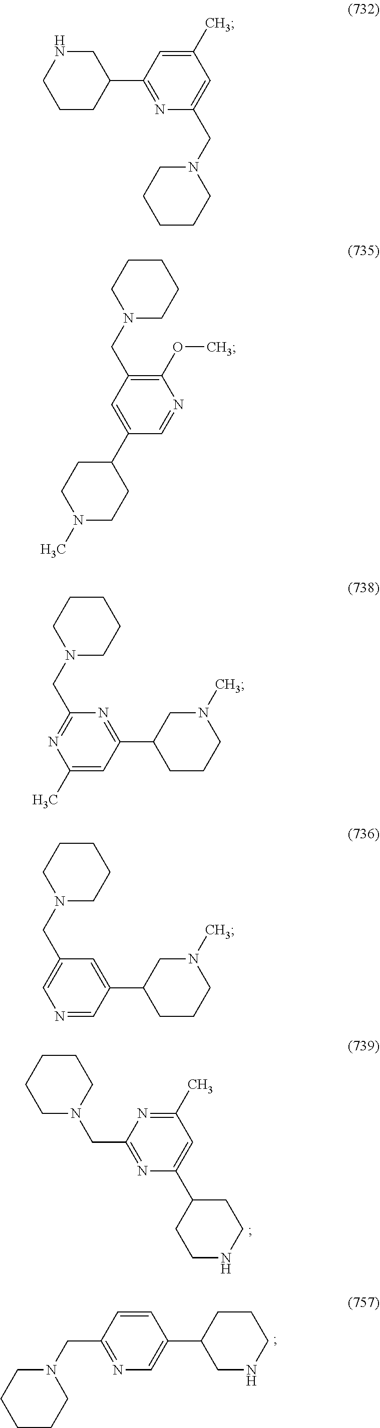

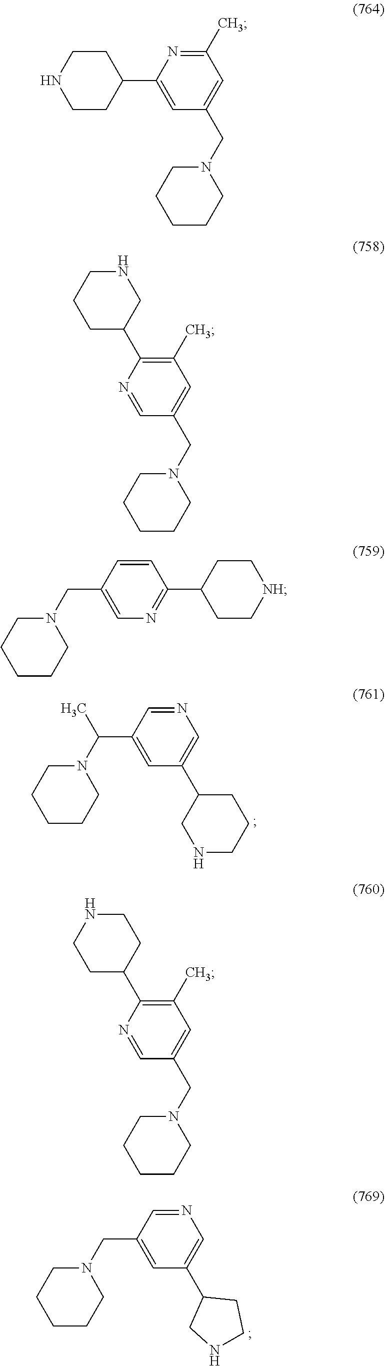

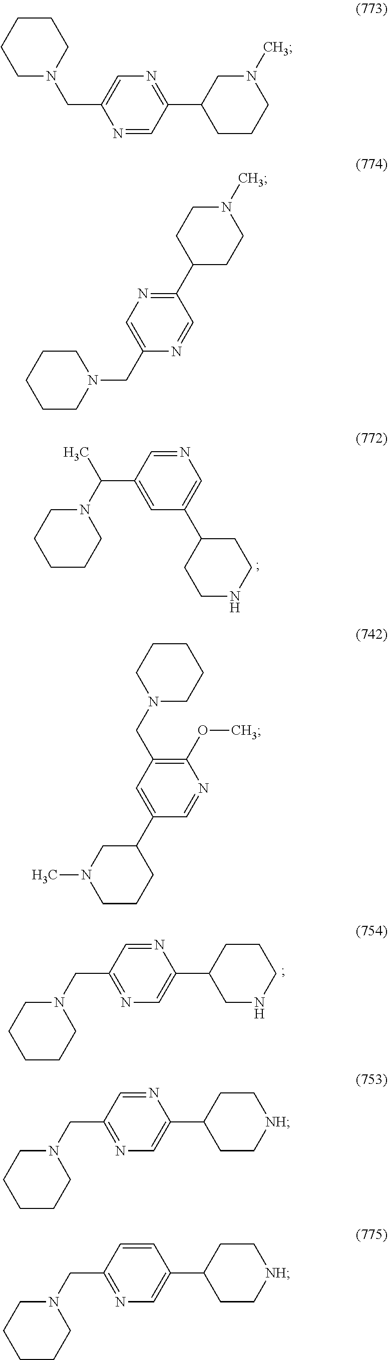

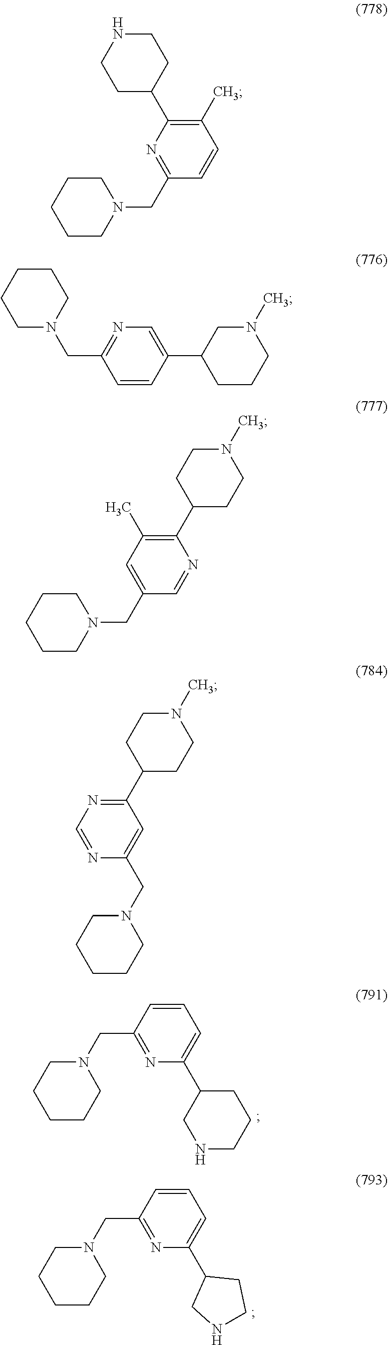

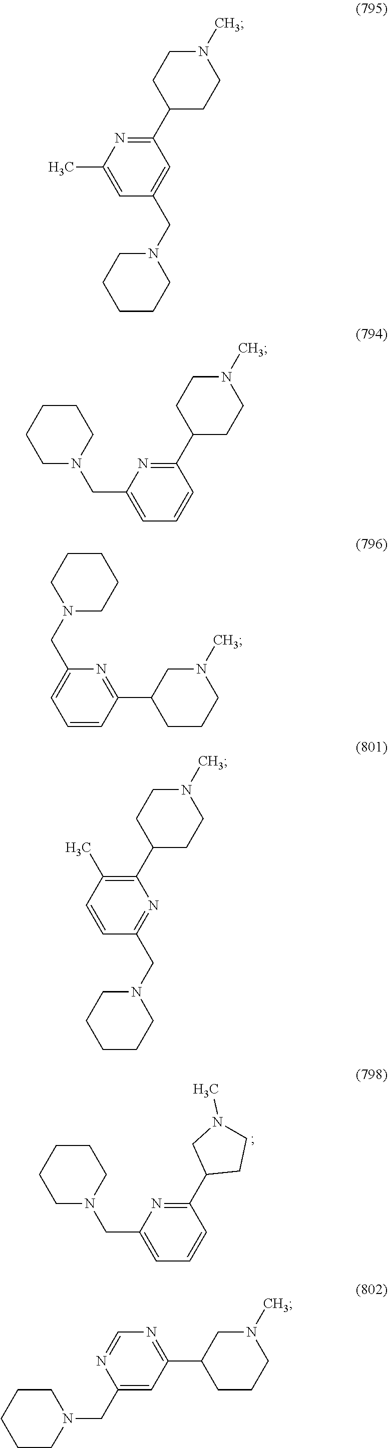

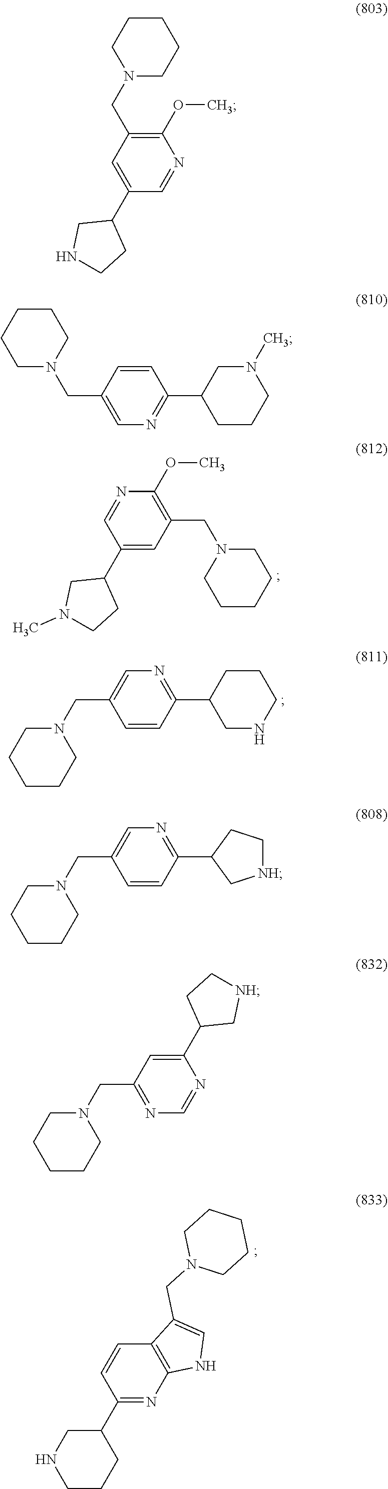

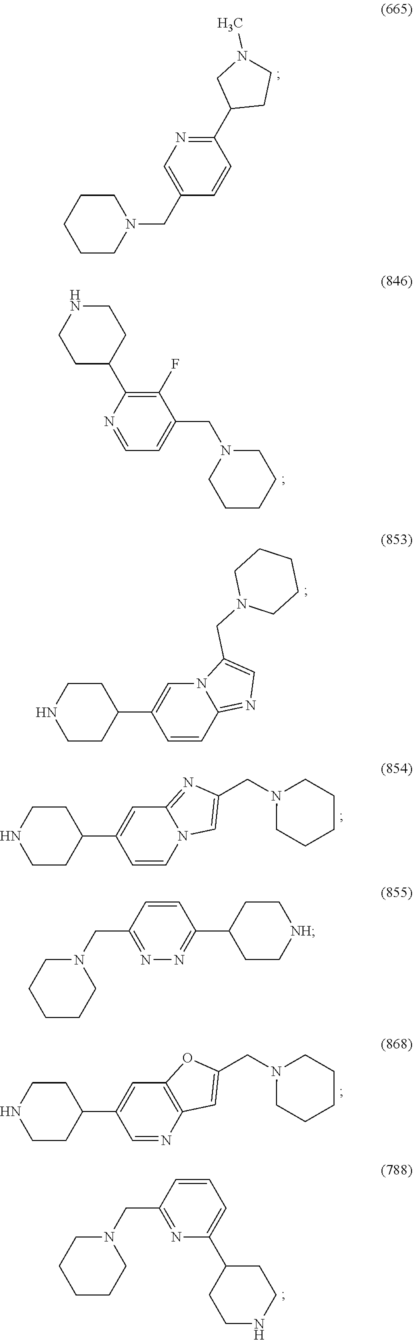

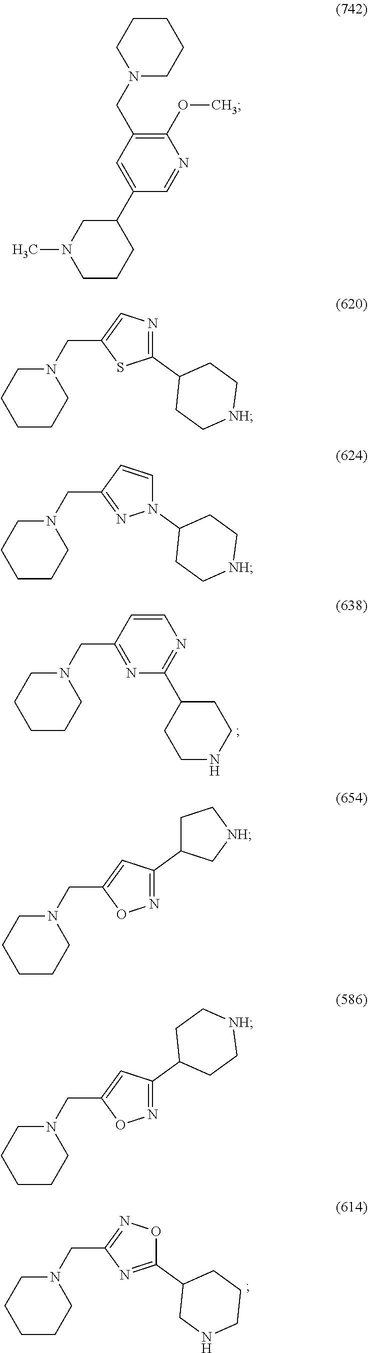

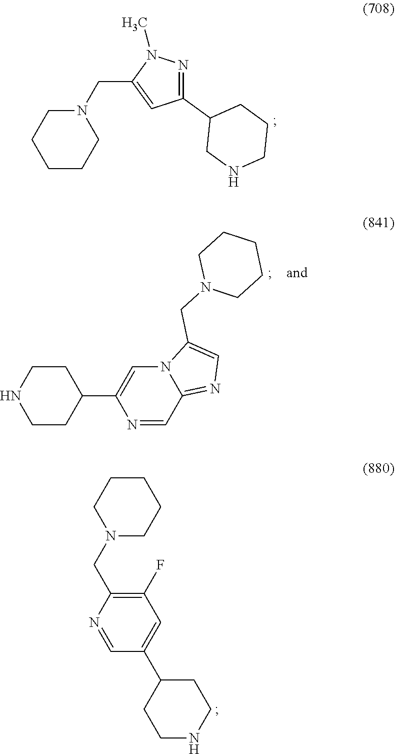 [SMILES: Cn1nc(C2CCCNC2)cc1CN1CCCCC1 ; c1nc2cnc(C3CCNCC3)cn2c1CN1CCCCC1 ; Fc1cc(C2CCNCC2)cnc1CN1CCCCC1]

and a pharmaceutically acceptable salt thereof.

### GBA Modulation

In one embodiment, the compound described herein increases glucocerebrosidase (GBA) enzyme levels and/or GBA enzyme activity.

In one embodiment, the compound is a GBA inducer. In one embodiment, the compound is provided for use in a method of increasing GBA levels and/or activity. In one embodiment, GBA activity is increased at least 1.5-fold, such as at least 2-fold, for example at least 2.5-fold, such as at least 3-fold. Hence, the compounds of the present disclosure are GBA inducers, i.e. capable of inducing increased GBA enzyme levels and/or activity. In one embodiment, the compound provided is a GBA inducer.

In one embodiment, the compound is provided for use in a method of increasing GBA levels and/or activity. This effect can be readily determined using the assay provided in Example 1.

In one embodiment, GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.

In one embodiment, the EC_{1.5} of the compound provided herein is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less, such as 9 μM or less, such as 8 μM or less, such as 7 μM or less, such as 6 μM or less, such as 5 μM or less, such as 4 μM or less, such as 3 μM or less, such as 2 μM or less, such as 1 μM or less.

In one embodiment, E max % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.

### Composition

In one embodiment, a pharmaceutical composition comprising a compound as defined herein is provided, and one or more pharmaceutically acceptable adjuvants, excipients, carriers, buffers and/or diluents.

### Treatment of Diseases

In one embodiment, a method for treating a disease in a subject is provided comprising administering a compound as defined herein to the subject, wherein the disease is associated with reduced GBA levels and/or activity.

In one embodiment, the disease is Parkinson's disease (PD).

In one embodiment, a method of increasing the GBA activity and/or levels is provided comprising contacting GBA with a compound as defined herein.

In one embodiment, the use of a compound as defined herein is provided, for the manufacture of a medicament for the treatment of Parkinson's disease (PD).

### Items

1. A compound of formula (I),

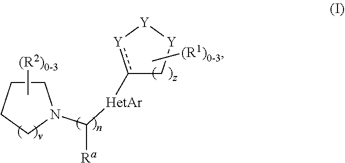

or a pharmaceutically acceptable salt or solvate thereof; wherein
   HetAr is a monocyclic or bicyclic heteroaryl comprising from 1 to 4 nitrogen atoms, from 0 to 2 oxygen atoms; and from 0 to 1 sulphur atoms in the ring with the remainder of ring-atoms being carbon; wherein HetAr is optionally substituted;
   each Y is independently selected from the group: N, NH, CH, and CH₂;
   z is 1 or 2;
   n is 1 or 2;
   v is 1 or 2;
   each R^{a} is independently selected from the group consisting of: hydrogen, alkyl, halogen, hydroxy, alkoxy, amino, amido, acyl, cycloalkyl, and heterocycloalkyl;
   each R¹ is independently selected from the group consisting of: hydrogen, alkyl, halogen, hydroxy, alkoxy, amino, amido, acyl, cycloalkyl, and heterocycloalkyl;
   each R₂ is independently selected from the group consisting of: hydrogen, alkyl, halogen, hydroxy, alkoxy, amino, amido, acyl, cycloalkyl, and heterocycloalkyl; and
   “ [symbol] ” is a single bond or a double bond.
2. A compound of formula (I),

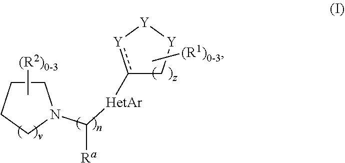

or a pharmaceutically acceptable salt or solvate thereof; wherein
   HetAr is a monocyclic or bicyclic heteroaryl comprising from 1 to 4 nitrogen atoms and from 0 to 2 oxygen atoms in the ring with the remainder of ring-atoms being carbon;
   each Y is independently selected from the group: N, NH, CH, and CH₂;
   z is 1 or 2;
   n is 1 or 2;
   v is 1 or 2;
   each R^{a} is independently selected from the group consisting of: hydrogen, alkyl, halogen, hydroxy, alkoxy, amino, amido, acyl, cycloalkyl, and heterocycloalkyl;
   each R¹ is independently selected from the group consisting of: hydrogen, alkyl, halogen, hydroxy, alkoxy, amino, amido, acyl, cycloalkyl, and heterocycloalkyl;
   each R² is independently selected from the group consisting of: hydrogen, alkyl, halogen, hydroxy, alkoxy, amino, amido, acyl, cycloalkyl, and heterocycloalkyl; and
   “ [symbol] ” is a single bond or a double bond.
3. The compound according to any one of the preceding items, wherein HetAr is a monocyclic heteroaryl.
4. The compound according to any one of the preceding items, wherein HetAr is a monocyclic heteroaryl comprising 1, 2, 3, or 4 nitrogen atoms in the ring, for example 1 or 2 nitrogen atoms.
5. The compound according to any one of the preceding items, wherein HetAr is a monocyclic heteroaryl comprising 0, 1, or 2 oxygen atoms in the ring.
6. The compound according to any one of the preceding items, wherein HetAr is a bicyclic heteroaryl.
7. The compound according to any one of the preceding items, wherein HetAr is a bicyclic heteroaryl comprising 1, 2 or 3 nitrogen atoms in the ring.
8. The compound according to any one of the preceding items, wherein HetAr is a bicyclic heteroaryl comprising 0, 1, or 2 oxygen atoms in the ring.
9. The compound according to any one of the preceding items, wherein HetAr comprises 5-10 ring atoms.
10. The compound according to any one of the preceding items, wherein the compound is of formula (IIa) or formula (IIb),

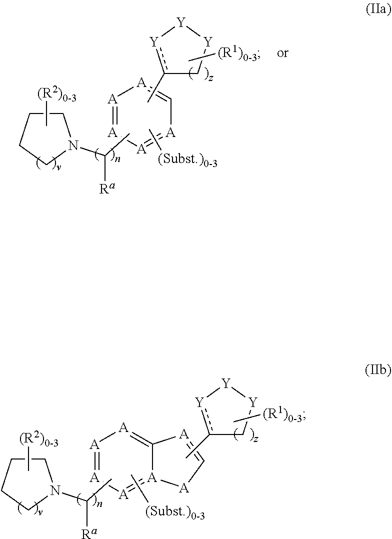

wherein each A is independently selected from the group: N, NH, C, CH, and CH₂;
   and wherein each Subst is independently selected from the group consisting of hydrogen, halogen, hydroxyl, alkoxy, amino, amido, alkyl, and cycloalkyl.
11. The compound according to any one of the preceding items, wherein the compound is of formula (IIIa) or formula (IIIb),

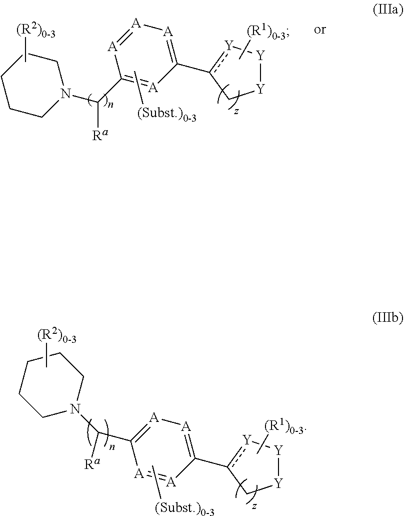

12. The compound according to any one of the preceding items, wherein “ [symbol] ” is a single bond.
13. The compound according to any one of the preceding items, wherein “ [symbol] ” is a double bond.
14. The compound according to any one of the preceding items, wherein n is 1.
15. The compound according to any one of the preceding items, wherein R^{a} is hydrogen, C₁₋₆ alkyl, or halogen.
16. The compound according to any one of the preceding items, wherein R^{a} is hydrogen or methyl.
17. The compound according to any one of the preceding items, wherein n is 1 and wherein R^{a} is hydrogen, C₁₋₆ alkyl, or halogen.
18. The compound according to any one of the preceding items, wherein n is 1 and wherein R^{a} is hydrogen or methyl.
19. The compound according to any one of the preceding items, wherein v is 2.
20. The compound according to any one of the preceding items, wherein one R¹ is hydrogen or C₁₋₆ alkyl.
21. The compound according to any one of the preceding items, wherein one Y is NH and each other Y is independently CH or CH₂.
22. The compound according to any one of the preceding items, wherein one Y is N which is bound to one R₁; and wherein each other Y is independently CH or CH₂.
23. The compound according to any one of the preceding items, wherein one Y is N which is bound to one R₁ which is hydrogen or C₁₋₆ alkyl; and wherein each other Y is independently CH or CH₂.
24. The compound according to any one of the preceding items, wherein R₂ is absent.
25. The compound according to any one of the preceding items, wherein the compound comprises one or two R₂ independently selected from the group consisting of: halogen and alkyl.
26. The compound according to any one of the preceding items, wherein the compound comprises one R₂ which is a C₁₋₆ alkyl.
27. The compound according to any one of the preceding items, wherein the compound comprises one R₂ which is halogen, such as fluorine.
28. The compound according to any one of the preceding items, wherein each Subst is independently selected from the group consisting of: hydrogen, C₁₋₆ alkyl, and C₁₋₆ alkoxy.
29. The compound according to any one of the preceding items, wherein the compound comprises one Subst selected from the group consisting of: hydrogen, C₁₋₆ alkyl, and C₁₋₆ alkoxy.
30. The compound according to any one of the preceding items, wherein two A are each N or NH, and the remainder of A are each C, CH or CH₂.
31. The compound according to any one of the preceding items, wherein one A is N or NH, and the remainder of A are each C, CH or CH₂.
32. The compound according to any one of the preceding items, wherein the compound is of formula (IIb) and one A is O, and one A is N or NH, and the remainder of A are each C, CH or CH₂.
33. The compound according to any one of the preceding items, wherein the compound is selected from the group consisting of:

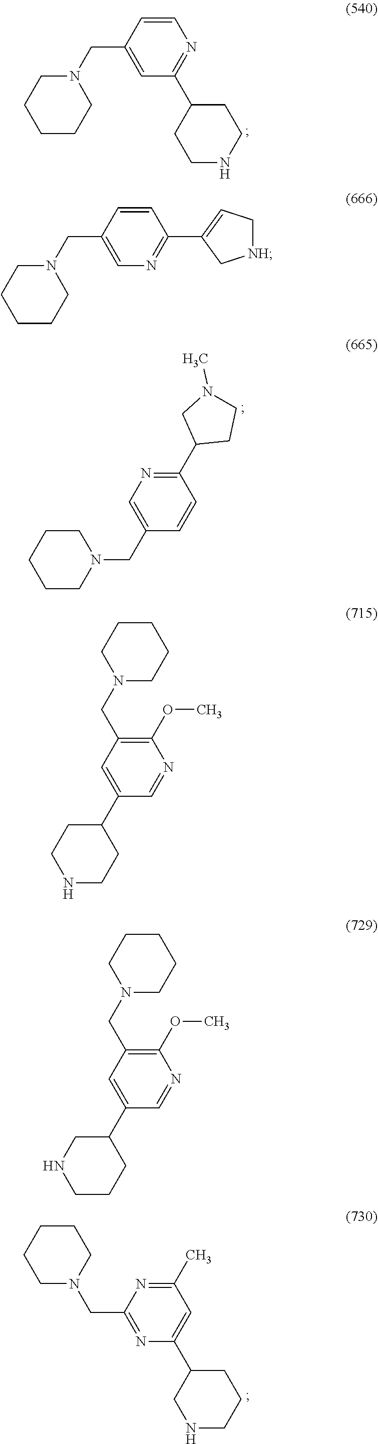

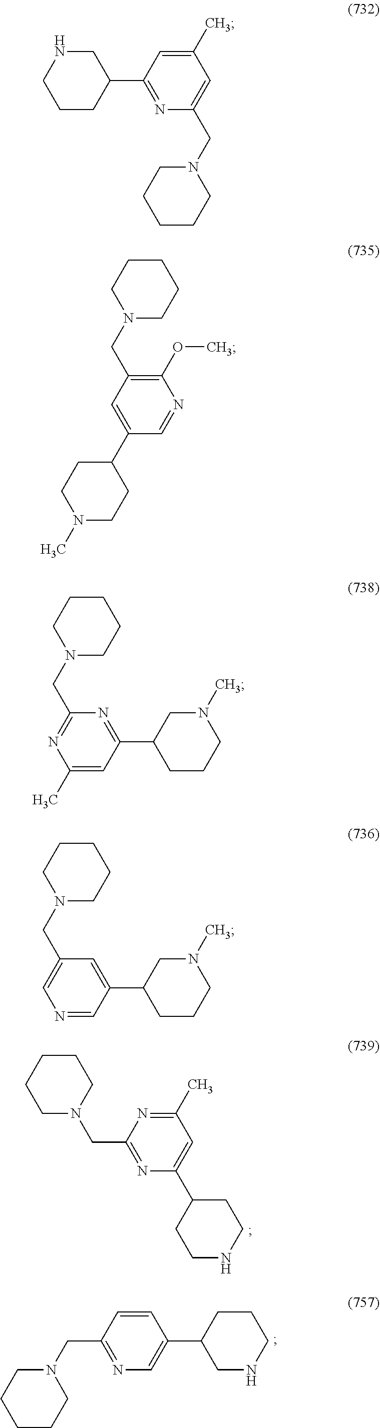

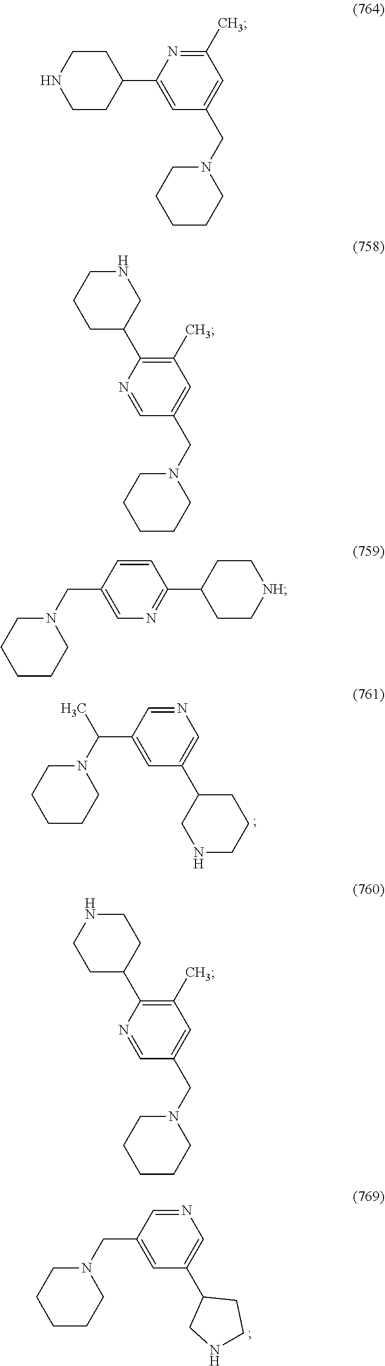

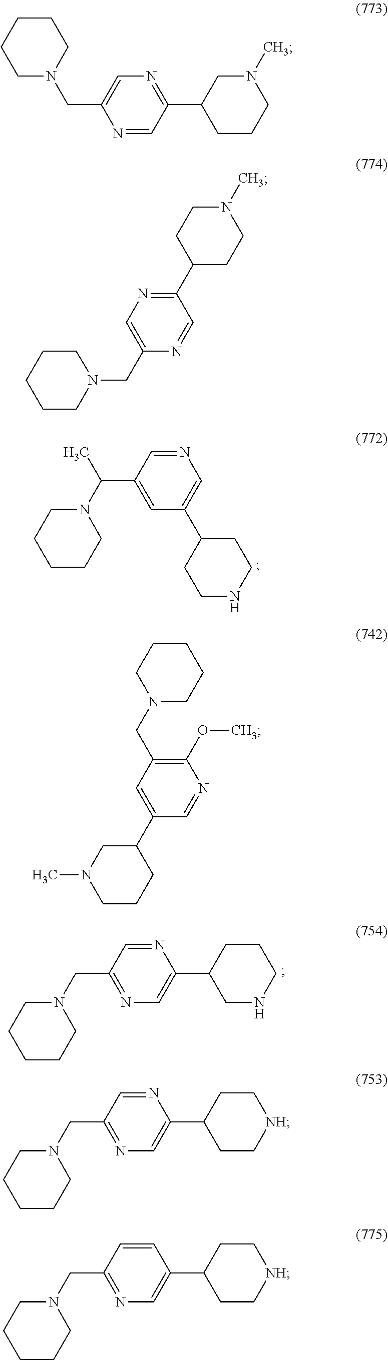

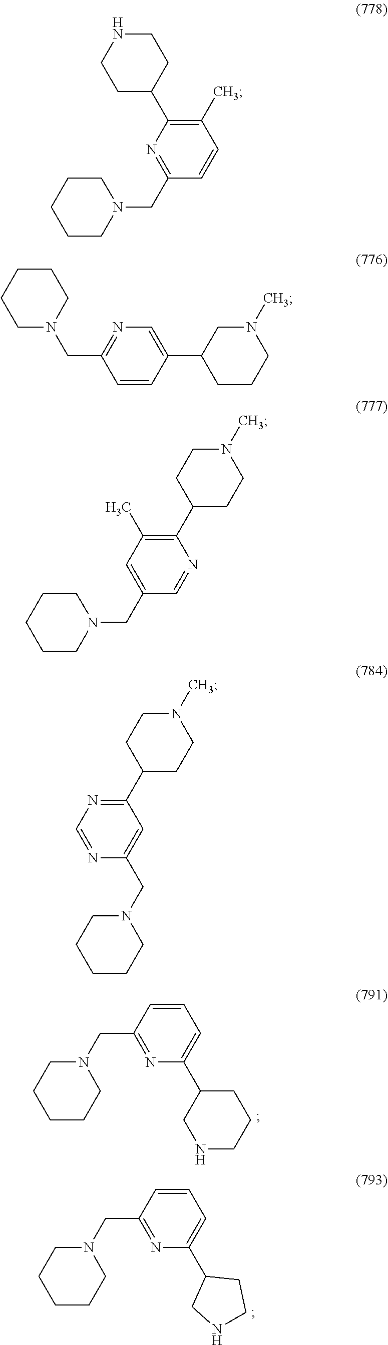

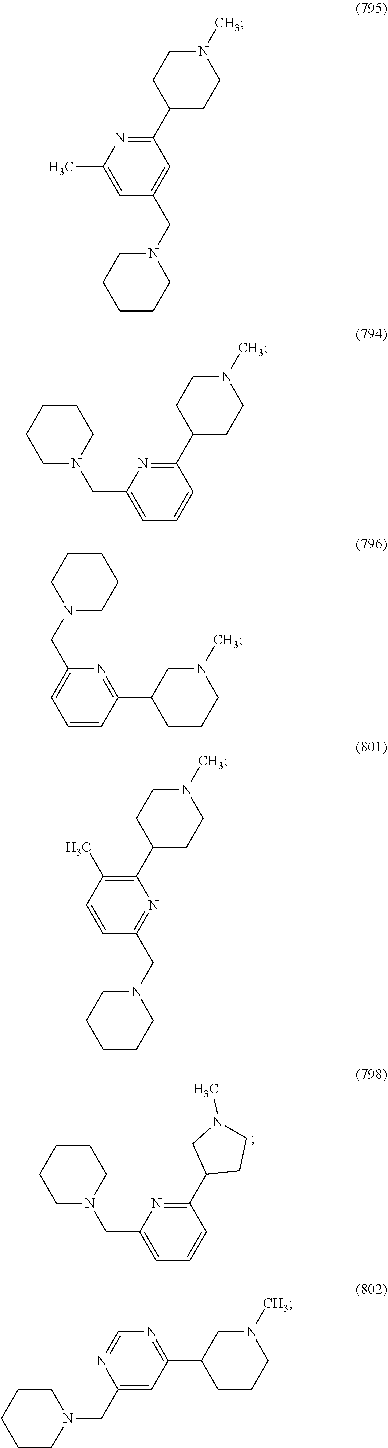

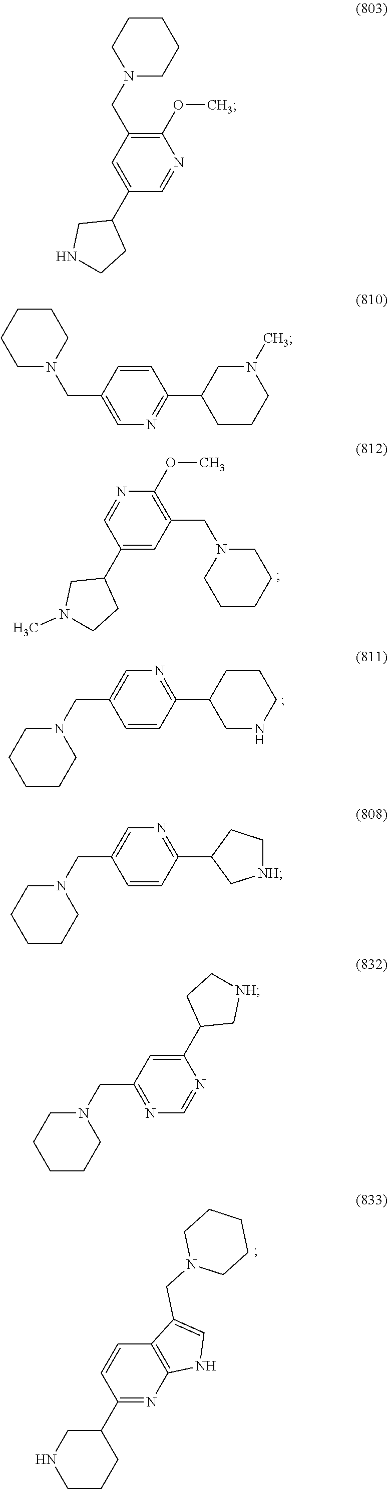

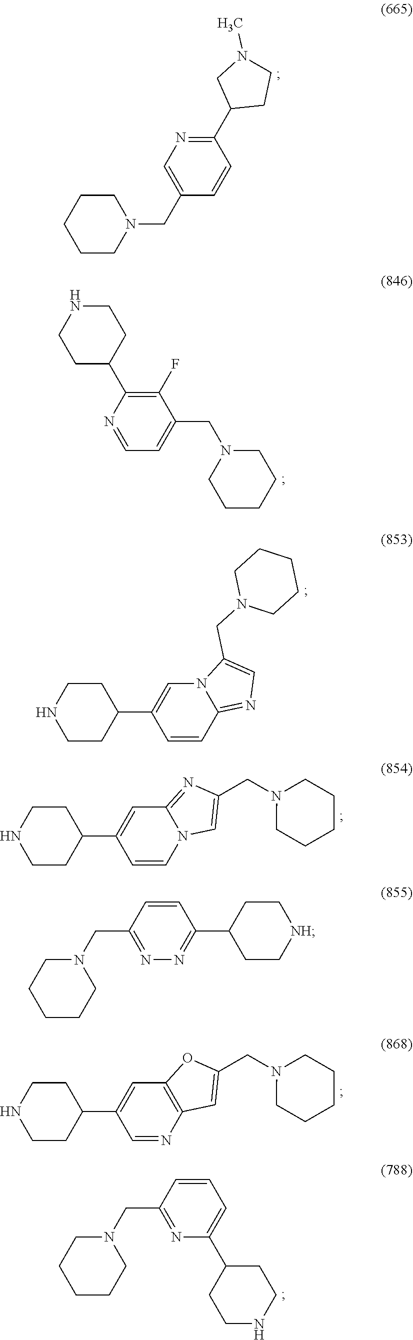

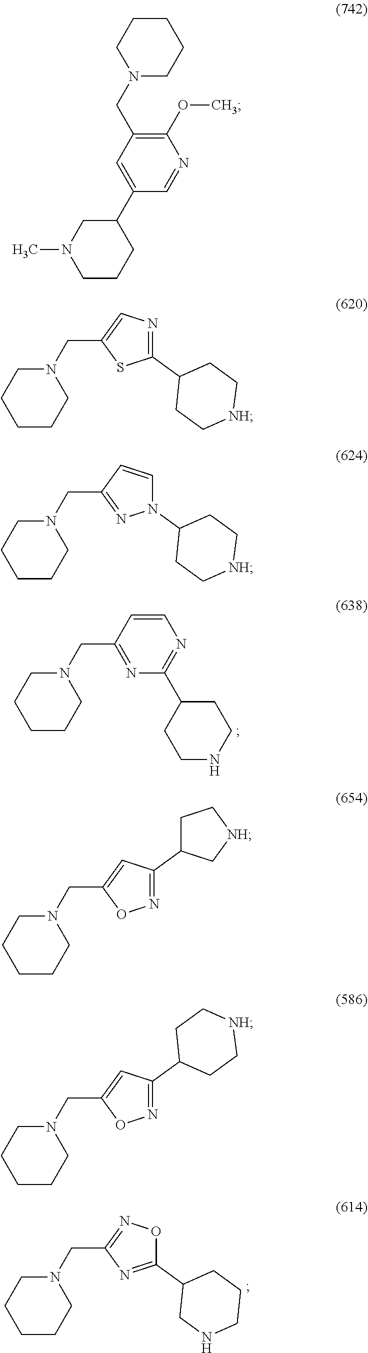

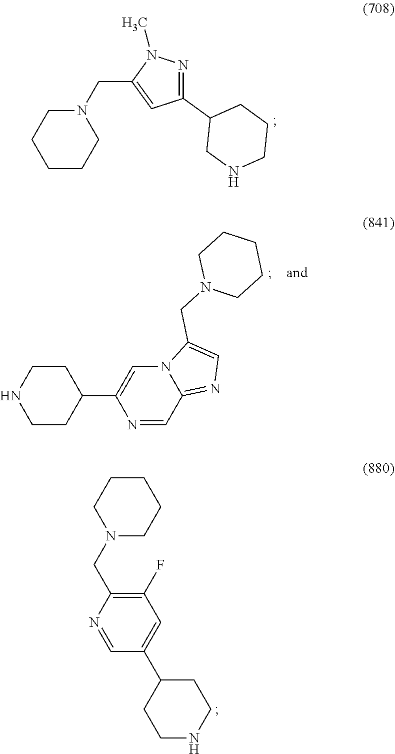 [SMILES: Cn1nc(C2CCCNC2)cc1CN1CCCCC1 ; c1nc2cnc(C3CCNCC3)cn2c1CN1CCCCC1 ; Fc1cc(C2CCNCC2)cnc1CN1CCCCC1]

and a pharmaceutically acceptable salt thereof.
34. The compound according to any one of the preceding items, wherein the compound increases glucocerebrosidase (GBA) enzyme levels and/or GBA enzyme activity.
35. The compound according to any one of the preceding items, wherein the compound is a GBA inducer.
36. The compound according to any one of the preceding items, for use in a method of increasing GBA levels and/or activity.
37. The compound for use according to any one of the preceding items, wherein said GBA activity is increased at least 1.5-fold, such as at least 2-fold, for example at least 2.5-fold, such as at least 3-fold.
38. The compound for use according to any one of the preceding items, wherein said GBA activity is increased to 50% or more of hypothetical wild-type levels, such as 50-60%, such as 60-70%, such as 70-80%, such as 80-90%, such as 90-100%, such as 100-110%, such as 110-120%, such as 120-130%, such as 130-140%, such as 140-150% of hypothetical wild-type levels.
39. The compound for use according to any one of the preceding items, wherein the EC_{1.5} of the compound is 150 μM or less, such as 140 μM or less, such as 130 μM or less, such as 120 μM or less, such as 110 μM or less, such as 100 μM or less, such as 90 μM or less, such as 80 μM or less, such as 70 μM or less, such as 60 μM or less, preferably wherein the EC_{1.5} is 50 μM or less, such as 40 μM or less, such as 30 μM or less, such as 20 μM or less, such as 10 μM or less, such as 9 μM or less, such as 8 μM or less, such as 7 μM or less, such as 6 μM or less, such as 5 μM or less, such as 4 μM or less, such as 3 μM or less, such as 2 μM or less, such as 1 μM or less.
40. The compound for use according to any one of the preceding items, wherein the E max % of the compound is 80% or more, such as 100% or more, such as 120% or more, such as 140% or more, such as 160% or more, such as 180% or more, such as 200% or more, such as 220% or more, such as 240% or more, such as 260% or more, such as 280% or more, such as 300% or more.
41. A pharmaceutical composition comprising a compound as defined in any one of the preceding items, and one or more pharmaceutically acceptable adjuvants, excipients, carriers, buffers and/or diluents.
42. A method for treating a disease in a subject comprising administering a compound as defined in any one of the preceding items to the subject, wherein the disease is associated with reduced GBA levels and/or activity.
43. The method according to any one of the preceding items, wherein the disease is Parkinson's disease (PD).
44. A method of increasing the GBA activity and/or levels comprising contacting GBA with a compound as defined in any one of the preceding items.
45. Use of a compound as defined in any one of the preceding items, for the manufacture of a medicament for the treatment of Parkinson's disease (PD).

### EXAMPLES

### Example 1: Determination of Potencies and Efficacies of Pyridines Using GCase Assay

### Materials

Human fibroblast cell line GM10915 harboring the L444P GBA mutation was obtained from Coriell Biorepositories.

All chemicals (Glacial acetic acid, Glycine, 4-Methylumbelliferyl b-D-glucopyranoside (4-MUG), Sodium acetate trihydrate, Sodium hydroxide, Crystal violet, SDS, Ammonium hydroxide) were obtained from Sigma-Aldrich (Denmark) Compounds tested for GCase activity were dissolved in H₂O or DMSO.

### Methods

The GM10915 cell line was cultured under standard cell culture conditions (37° C. and 5% CO₂) in complete DMEM medium supplemented with nonessential amino acids (NEAA), 1% Pen-Strep and 12% FCS. Cells were seeded at a density of 10⁴ cells/well in 100 μL complete medium in one black 96-well plate for glucosylceramidase (GCase) activity measurement and in one clear 96-well plate for crystal violet staining to correct for cell density. Crystal violet staining is performed to obtain quantitative information about the relative density of cells adhering to multi-wells plates.

### Assay of GCase Activity

The assay was adapted from Sawkar et al (2002) and briefly described in the following. The day after seeding of cells, the medium was replaced with fresh medium containing the compounds to be tested. Compounds were tested in duplicate and in an 8-point diluted dose range to obtain a dose response. Cells were exposed with compounds for five days. Fresh compound was added every 2-3 days. PBS was included to define the basal level of GCase activity.

Cells were washed three times with 200 μL PBS per well and 50 μL of 2.5 mM 4-MUG buffer (4-MUG dissolved in 0.2 M acetate buffer pH 4.0) was added and the cells were incubated at 37° C., 5% CO₂ for 23 hours. The reaction was stopped by adding 150 μL 0.2 M glycine buffer (pH 10.8). Fluorescence was measured with a Varioskan® Flash reader (Thermo Scientific) at an excitation/emission setting of 365/445 nm.

### Crystal Violet Staining

Cells were treated with compounds in a parallel setup identical to the setup to test for GCase activity. At the end of compound treatment, cells were washed once with 200 μL PBS per well and 50 μL 0.1% w/v crystal violet (in H₂0) was added. Following 10 min. of incubation, the crystal violet solution was removed, and the cells were washed three times with 200 μL PBS and 100 μL 1% SDS was added to solubilize the stain.

The plate was agitated on an orbital shaker for 10-30 min. Absorbance (A) is measured at 570 nM using a Varioskan® Flash reader (Thermo Scientific).

### Calculations

The fluorescence signal (F) derived from the GCase measurement is normalized to the absorbance signal (A) derived from the crystal violet staining. The percent GCase activity resulting from compound treatment is calculated relative to the basal activity obtained from untreated cells.

$$\\ Percent GCase activity = 100 * \frac{{( F / A )}_{compound}}{{( F / A )}_{untreated}}$$

The potency, EC_{1.5}, is determined based on the dose response effects of the compounds as the concentration where “Percent GCase activity”=150% corresponding to at 1.5-fold induction of GCase activity. Maximal effect of compounds (E max) is determined from the dose response effects as the maximum “Percent GCase activity” achieved in the dose range tested.

### Results

The GBA potencies and E max were determined as described above in the present example and the results are shown in Table 1 below.

| ID | Structure | GBA EC_{1.5} (μM) | GBA Emax (%) |
|---|---|---|---|
| Ambroxol reference | 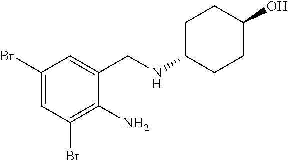 [SMILES: Nc1c(Br)cc(Br)cc1CN[C@H]1CC[C@H](O)CC1] | >20* *The 1.5-fold induction criteria was not reached within the tested concentration range | 127 |
| LTI-291 reference | 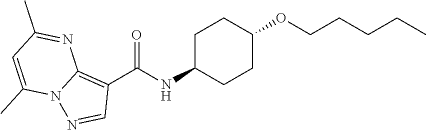 [SMILES: CCCCCO[C@H]1CC[C@H](NC(=O)c2cnn3c(C)cc(C)nc23)CC1] | 112 | 232 |
| 540 | 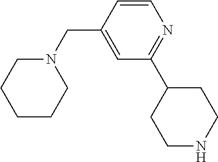 [SMILES: c1cc(CN2CCCCC2)cc(C2CCNCC2)n1] | 5.4 | 177 |
| 666 | 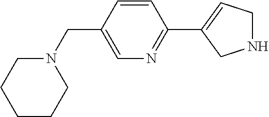 [SMILES: C1=C(c2ccc(CN3CCCCC3)cn2)CNC1] | 9.2 | 253 |
| 665 | 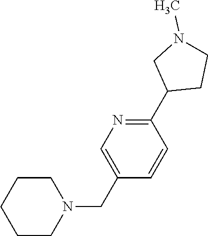 [SMILES: CN1CCC(c2ccc(CN3CCCCC3)cn2)C1] | 2.7 | 194 |
| 715 | 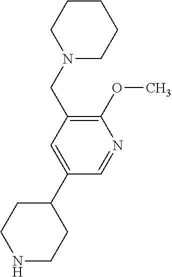 [SMILES: COc1ncc(C2CCNCC2)cc1CN1CCCCC1] | 24.8 | 160 |
| 729 | 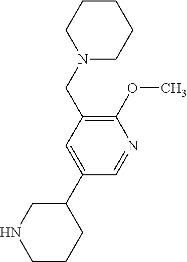 [SMILES: COc1ncc(C2CCCNC2)cc1CN1CCCCC1] | 2.7 | 223 |
| 730 | 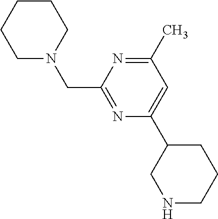 [SMILES: Cc1cc(C2CCCNC2)nc(CN2CCCCC2)n1] | 5.0 | 234 |
| 732 | 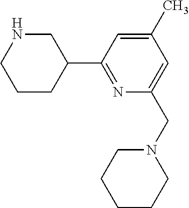 [SMILES: Cc1cc(CN2CCCCC2)nc(C2CCCNC2)c1] | 3.2 | 200 |
| 735 | 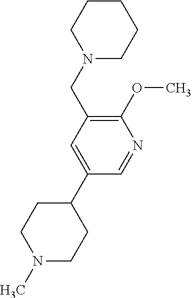 [SMILES: COc1ncc(C2CCN(C)CC2)cc1CN1CCCCC1] | 2.3 | 212 |
| 738 | 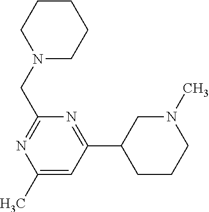 [SMILES: Cc1cc(C2CCCN(C)C2)nc(CN2CCCCC2)n1] | 5.4 | 248 |
| 736 | 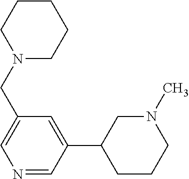 [SMILES: CN1CCCC(c2cncc(CN3CCCCC3)c2)C1] | 3.7 | 216 |
| 739 | 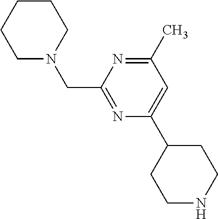 [SMILES: Cc1cc(C2CCNCC2)nc(CN2CCCCC2)n1] | 30.8 | 176 |
| 757 | 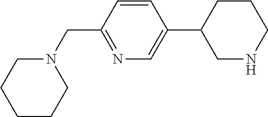 [SMILES: c1cc(CN2CCCCC2)ncc1C1CCCNC1] | 4.2 | 214 |
| 764 | 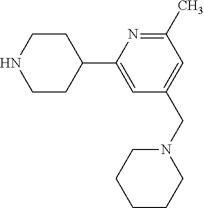 [SMILES: Cc1cc(CN2CCCCC2)cc(C2CCNCC2)n1] | 3.3 | 227 |
| 758 | 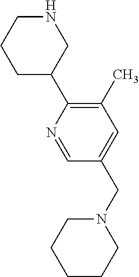 [SMILES: Cc1cc(CN2CCCCC2)cnc1C1CCCNC1] | 3.1 | 233 |
| 759 | 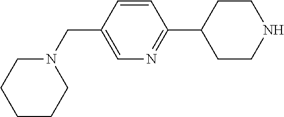 [SMILES: c1cc(C2CCNCC2)ncc1CN1CCCCC1] | 6.6 | 200 |
| 761 | 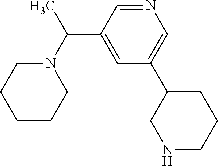 [SMILES: CC(c1cncc(C2CCCNC2)c1)N1CCCCC1] | 5.2 | 205 |
| 760 | 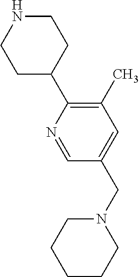 [SMILES: Cc1cc(CN2CCCCC2)cnc1C1CCNCC1] | 4.3 | 211 |
| 769 | 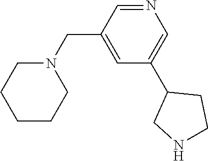 [SMILES: c1ncc(C2CCNC2)cc1CN1CCCCC1] | 18.9 | 205 |
| 773 | 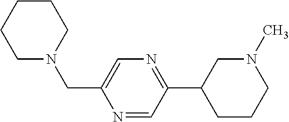 [SMILES: CN1CCCC(c2cnc(CN3CCCCC3)cn2)C1] | 3.9 | 236 |
| 774 | 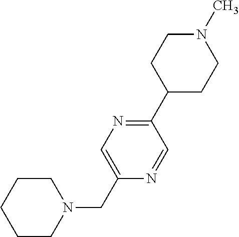 [SMILES: CN1CCC(c2cnc(CN3CCCCC3)cn2)CC1] | 9.7 | 198 |
| 772 | 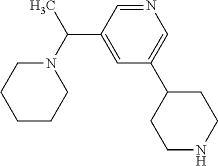 [SMILES: CC(c1cncc(C2CCNCC2)c1)N1CCCCC1] | 18.5 | 211 |
| 742 | 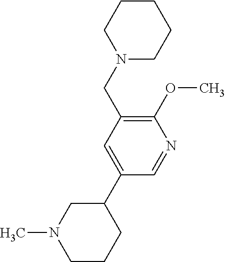 | 3.9 | 170 |
| 754 | 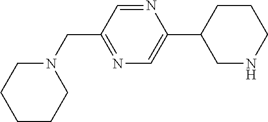 [SMILES: c1nc(C2CCCNC2)cnc1CN1CCCCC1] | 11.8 | 180 |
| 753 | 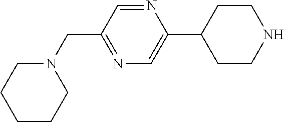 [SMILES: c1nc(C2CCNCC2)cnc1CN1CCCCC1] | 19.1 | 190 |
| 775 | 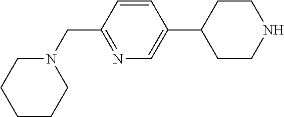 [SMILES: c1cc(CN2CCCCC2)ncc1C1CCNCC1] | 24.5 | 186 |
| 778 | 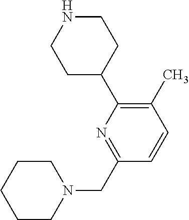 [SMILES: Cc1ccc(CN2CCCCC2)nc1C1CCNCC1] | 7.4 | 152 |
| 776 | 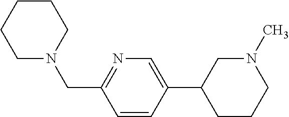 [SMILES: CN1CCCC(c2ccc(CN3CCCCC3)nc2)C1] | 4.3 | 200 |
| 777 | 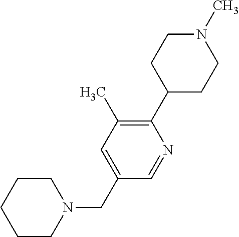 [SMILES: Cc1cc(CN2CCCCC2)cnc1C1CCN(C)CC1] | 6.9 | 200 |
| 784 | 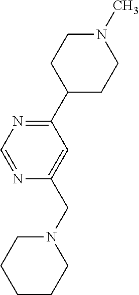 [SMILES: CN1CCC(c2cc(CN3CCCCC3)ncn2)CC1] | 6.5 | 155 |
| 791 | 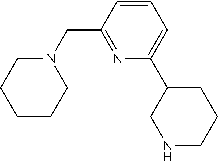 [SMILES: c1cc(CN2CCCCC2)nc(C2CCCNC2)c1] | 4.4 | 165 |
| 793 | 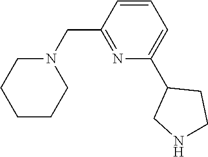 [SMILES: c1cc(CN2CCCCC2)nc(C2CCNC2)c1] | 29.2 | 165 |
| 795 | 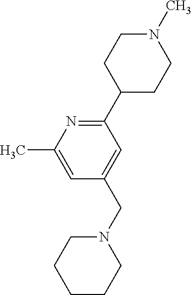 | 7.0 | 185 |
| 794 | 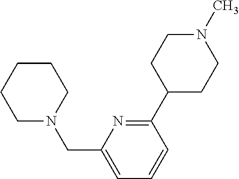 [SMILES: CN1CCC(c2cccc(CN3CCCCC3)n2)CC1] | 3.0 | 269 |
| 796 | 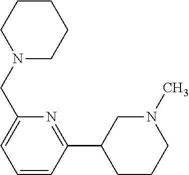 [SMILES: CN1CCCC(c2cccc(CN3CCCCC3)n2)C1] | 2.6 | 267 |
| 801 | 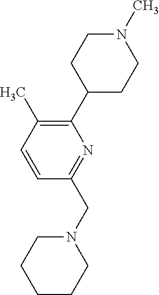 [SMILES: Cc1ccc(CN2CCCCC2)nc1C1CCN(C)CC1] | 3.9 | 173 |
| 798 | 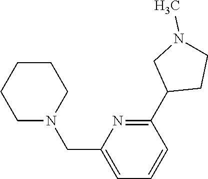 [SMILES: CN1CCC(c2cccc(CN3CCCCC3)n2)C1] | 2.5 | 250 |
| 802 | 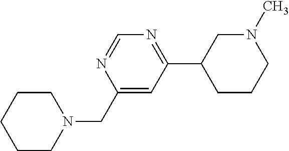 [SMILES: CN1CCCC(c2cc(CN3CCCCC3)ncn2)C1] | 8.8 | 256 |
| 803 | 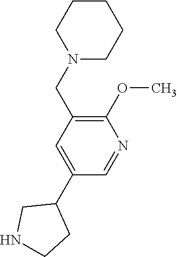 [SMILES: COc1ncc(C2CCNC2)cc1CN1CCCCC1] | 6.0 | 203 |
| 810 | 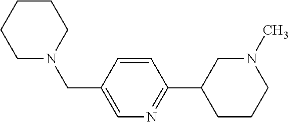 [SMILES: CN1CCCC(c2ccc(CN3CCCCC3)cn2)C1] | 4.9 | 199 |
| 812 | 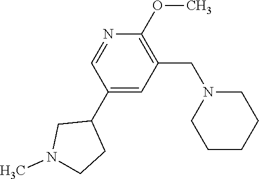 [SMILES: COc1ncc(C2CCN(C)C2)cc1CN1CCCCC1] | 6.7 | 255 |
| 811 | 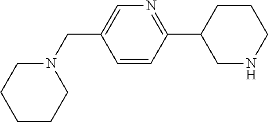 [SMILES: c1cc(C2CCCNC2)ncc1CN1CCCCC1] | 3.4 | 263 |
| 808 | 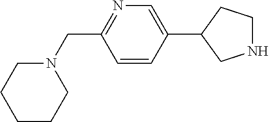 [SMILES: c1cc(CN2CCCCC2)ncc1C1CCNC1] | 28.6 | 175 |
| 832 | 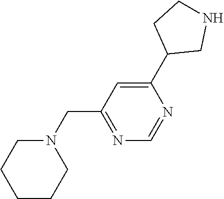 [SMILES: c1nc(CN2CCCCC2)cc(C2CCNC2)n1] | 18.0 | 175 |
| 833 | 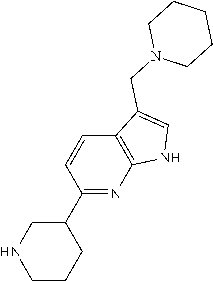 [SMILES: c1cc2c(CN3CCCCC3)c[nH]c2nc1C1CCCNC1] | 12.1 | 203 |
| 665 | 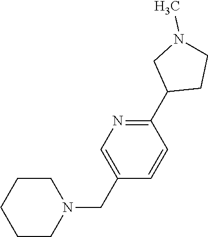 [SMILES: CN1CCC(c2ccc(CN3CCCCC3)cn2)C1] | 4.7 | 243 |
| 846 | 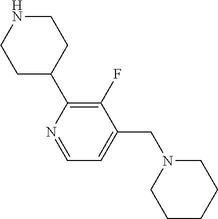 [SMILES: Fc1c(CN2CCCCC2)ccnc1C1CCNCC1] | 2.8 | 245 |
| 853 | 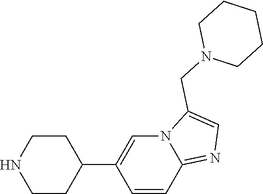 [SMILES: c1cc2ncc(CN3CCCCC3)n2cc1C1CCNCC1] | 8.0 | 193 |
| 854 | 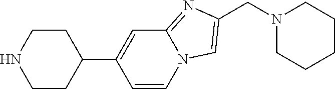 [SMILES: c1cn2cc(CN3CCCCC3)nc2cc1C1CCNCC1] | 9.4 | 194 |
| 855 | 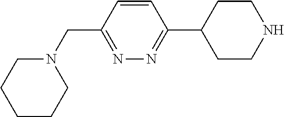 [SMILES: c1cc(C2CCNCC2)nnc1CN1CCCCC1] | 43.3 | 163 |
| 868 | 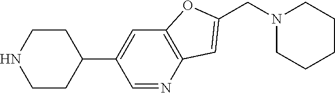 [SMILES: c1nc2cc(CN3CCCCC3)oc2cc1C1CCNCC1] | 6.5 | 204 |
| 788 | 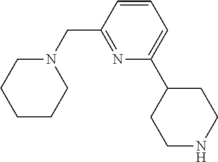 [SMILES: c1cc(CN2CCCCC2)nc(C2CCNCC2)c1] | 8.0 | 278 |
| 742 | 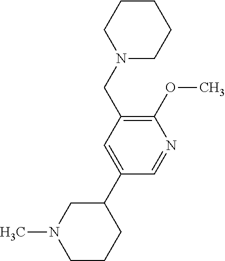 | 3.8 | 193 |
| 880 | 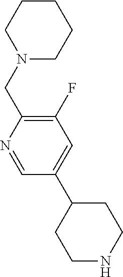 [SMILES: Fc1cc(C2CCNCC2)cnc1CN1CCCCC1] | 4.3 | 237 |
| 620 | 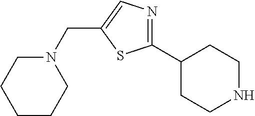 [SMILES: c1nc(C2CCNCC2)sc1CN1CCCCC1] | 3.5 | 236 |
| 624 | 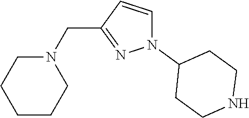 [SMILES: c1cn(C2CCNCC2)nc1CN1CCCCC1] | 21 | 189 |
| 708 | 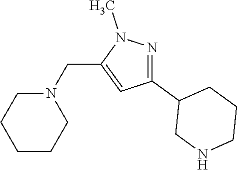 [SMILES: Cn1nc(C2CCCNC2)cc1CN1CCCCC1] | 16 | 205 |
| 737 | 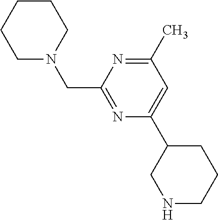 [SMILES: Cc1cc(C2CCCNC2)nc(CN2CCCCC2)n1] | 21.5 | 203 |
| 614 | 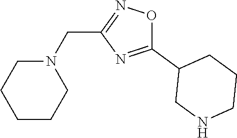 [SMILES: C1CCN(Cc2noc(C3CCCNC3)n2)CC1] | 46 | 169 |
| 586 | 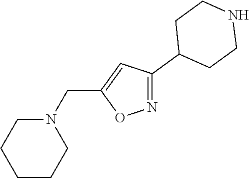 [SMILES: c1c(C2CCNCC2)noc1CN1CCCCC1] | 7.5 | 234 |
| 654 | 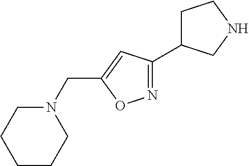 [SMILES: c1c(C2CCNC2)noc1CN1CCCCC1] | 4.9 | 381 |
| 638 | 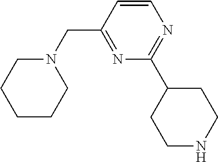 [SMILES: c1cc(CN2CCCCC2)nc(C2CCNCC2)n1] | 4.1 | 263 |
| 841 | 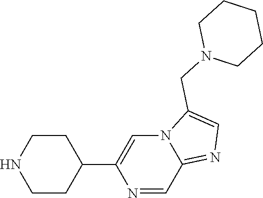 [SMILES: c1nc2cnc(C3CCNCC3)cn2c1CN1CCCCC1] | 35.9 | 181 |

1. A compound of formula (I), 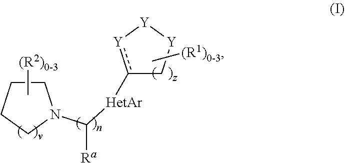
or a pharmaceutically acceptable salt or solvate thereof; wherein
HetAr is a monocyclic or bicyclic heteroaryl comprising from 1 to 4 nitrogen atoms, from 0 to 2 oxygen atoms; and from 0 to 1 sulphur atoms in the ring with the remainder of ring-atoms being carbon; wherein HetAr is optionally substituted;
each Y is independently selected from the group: N, NH, CH, and CH₂;
z is 1 or 2;
n is 1 or 2;
v is 1 or 2;
each R^{a} is independently selected from the group consisting of: hydrogen, alkyl, halogen, hydroxy, alkoxy, amino, amido, acyl, cycloalkyl, and heterocycloalkyl;
each R¹ is independently selected from the group consisting of: hydrogen, alkyl, halogen, hydroxy, alkoxy, amino, amido, acyl, cycloalkyl, and heterocycloalkyl;
each R₂ is independently selected from the group consisting of: hydrogen, alkyl, halogen, hydroxy, alkoxy, amino, amido, acyl, cycloalkyl, and heterocycloalkyl; and
“ [symbol] ” is a single bond or a double bond.

2. A compound of formula (I), 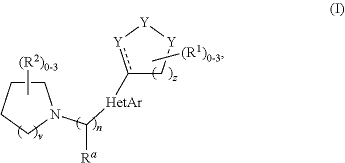
or a pharmaceutically acceptable salt or solvate thereof; wherein
HetAr is a monocyclic or bicyclic heteroaryl comprising from 1 to 4 nitrogen atoms and from 0 to 2 oxygen atoms in the ring with the remainder of ring-atoms being carbon;
each Y is independently selected from the group: N, NH, CH, and CH₂;
z is 1 or 2;
n is 1 or 2;
v is 1 or 2;
each R^{a} is independently selected from the group consisting of: hydrogen, alkyl, halogen, hydroxy, alkoxy, amino, amido, acyl, cycloalkyl, and heterocycloalkyl;
each R¹ is independently selected from the group consisting of: hydrogen, alkyl, halogen, hydroxy, alkoxy, amino, amido, acyl, cycloalkyl, and heterocycloalkyl;
each R² is independently selected from the group consisting of: hydrogen, alkyl, halogen, hydroxy, alkoxy, amino, amido, acyl, cycloalkyl, and heterocycloalkyl; and
“ [symbol] ” is a single bond or a double bond.

3. The compound according to any one of the preceding claims, wherein HetAr comprises 5-10 ring atoms.

4. The compound according to any one of the preceding claims, wherein HetAr is a monocyclic heteroaryl comprising 1, 2, 3, or 4 nitrogen atoms in the ring, for example 1 or 2 nitrogen atoms.

5. The compound according to any one of the preceding claims, wherein HetAr is a monocyclic heteroaryl comprising 0, 1, or 2 oxygen atoms in the ring.

6. The compound according to any one of claims 1-3, wherein HetAr is a bicyclic heteroaryl comprising 1, 2 or 3 nitrogen atoms in the ring.

7. The compound according to any one of claims 1-3 or 6, wherein HetAr is a bicyclic heteroaryl comprising 0, 1, or 2 oxygen atoms in the ring.

8. The compound according to any one of claims 1-5, wherein the compound is of formula (IIa) or formula (IIb), 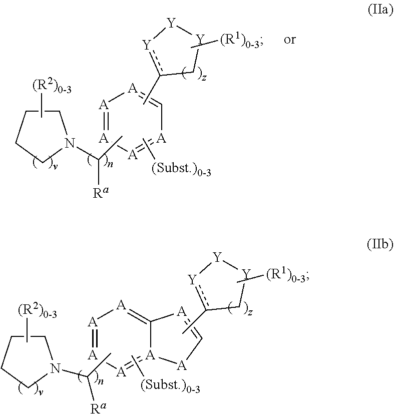
wherein each A is independently selected from the group: N, NH, C, CH, and CH₂;
and wherein each Subst is independently selected from the group consisting of hydrogen, halogen, hydroxyl, alkoxy, amino, amido, alkyl, and cycloalkyl.

9. The compound according to any one of claims 1-5, wherein the compound is of formula (IIIa) or formula (IIIb),

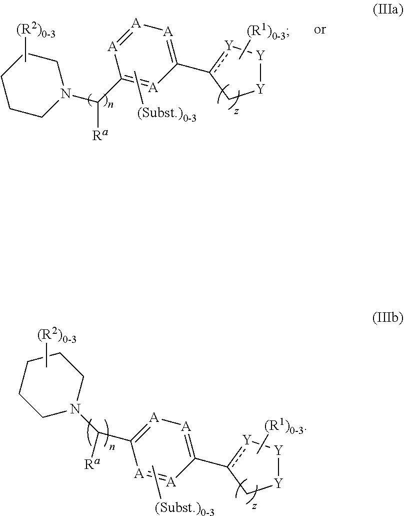

10. The compound according to any one of the preceding claims, wherein R^{a} is hydrogen, C₁₋₆ alkyl, or halogen.

11. The compound according to any one of the preceding claims, wherein two A are each N or NH, and the remainder of A are each C, CH or CH₂.

12. The compound according to any one of the preceding claims, wherein one A is N or NH, and the remainder of A are each C, CH or CH₂.

13. The compound according to any one of the preceding claims, wherein the compound is selected from the group consisting of: 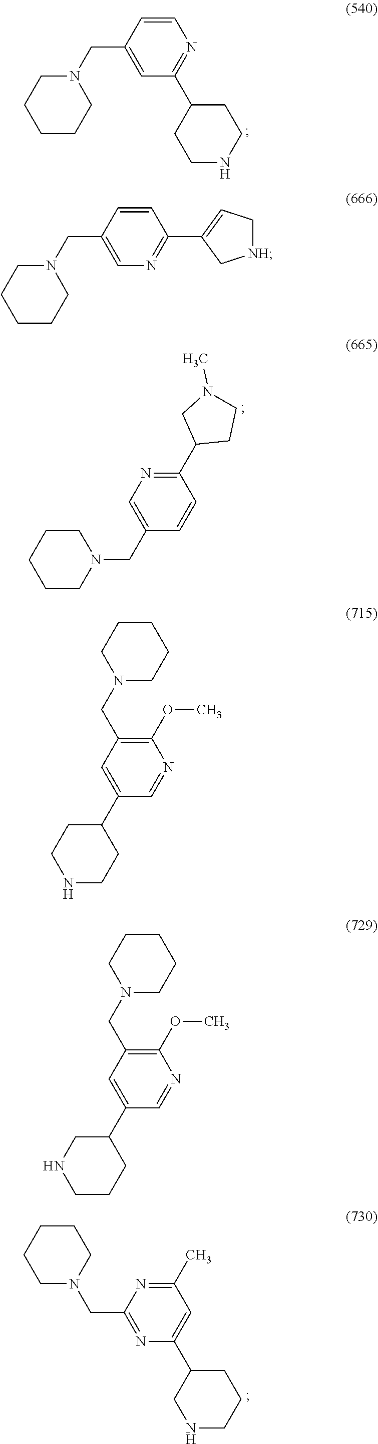 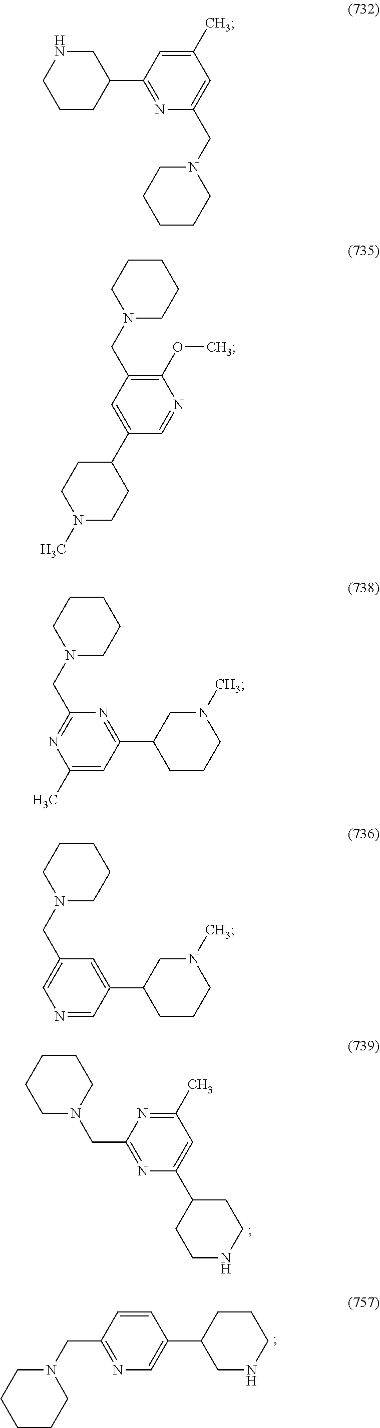 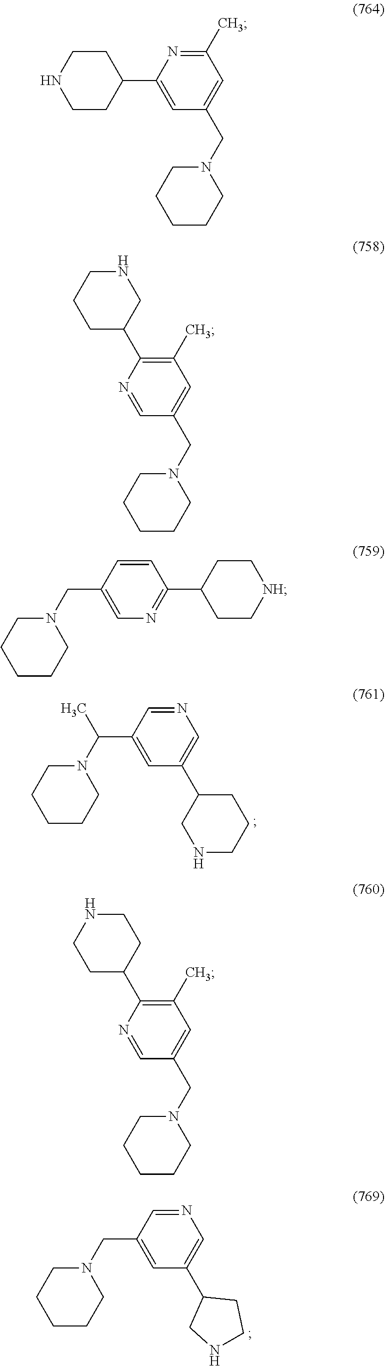 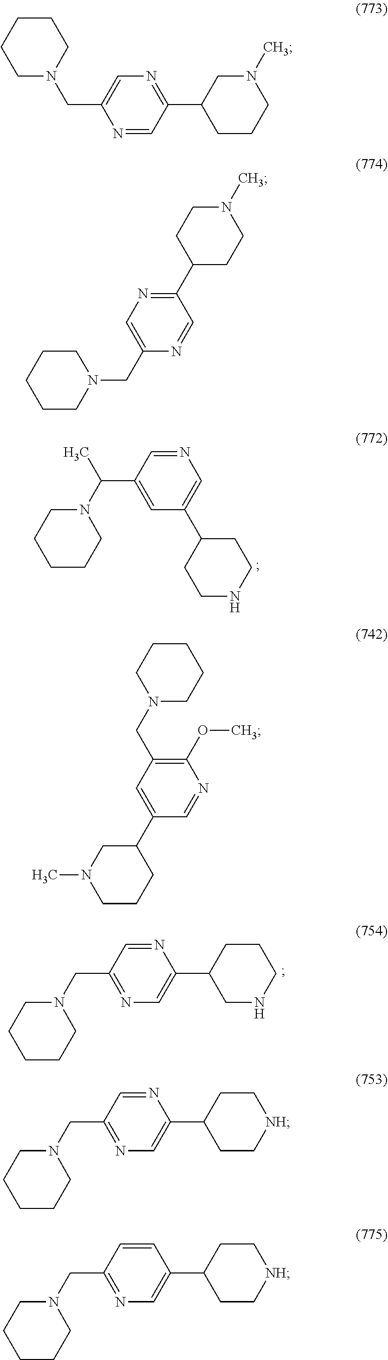 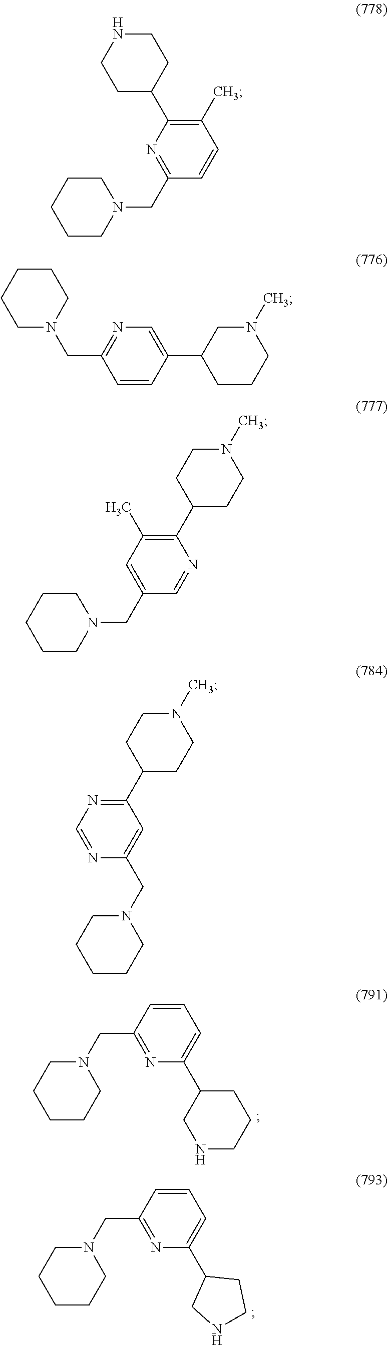 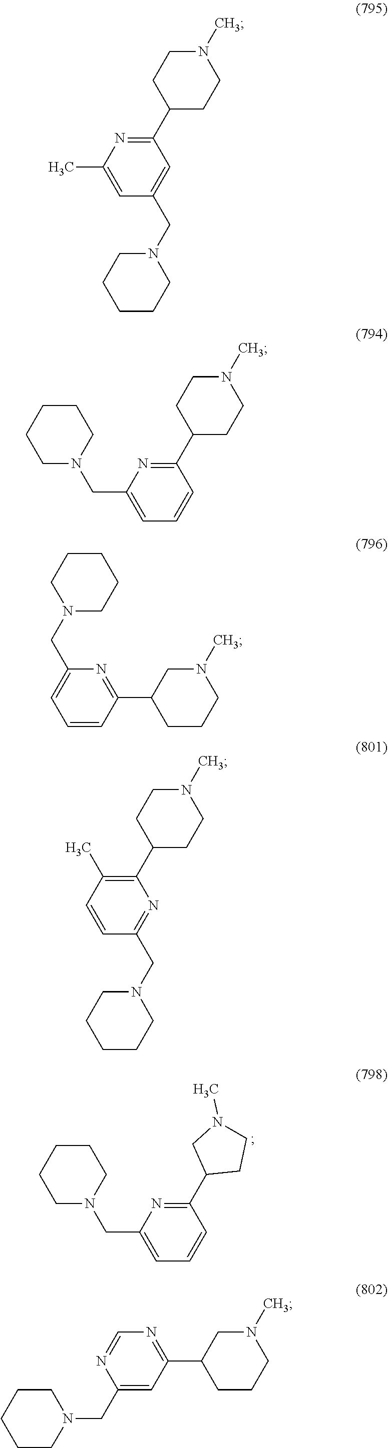 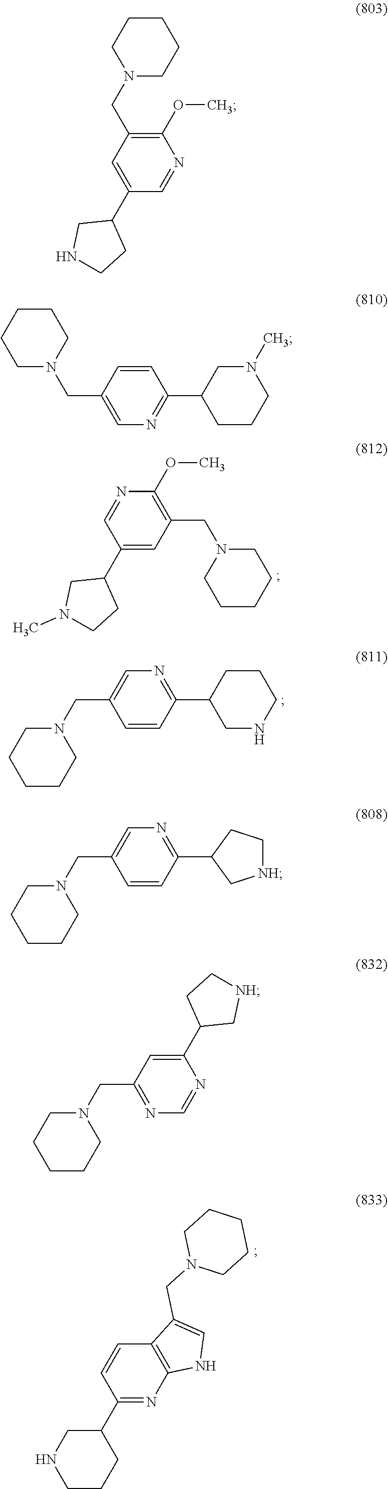 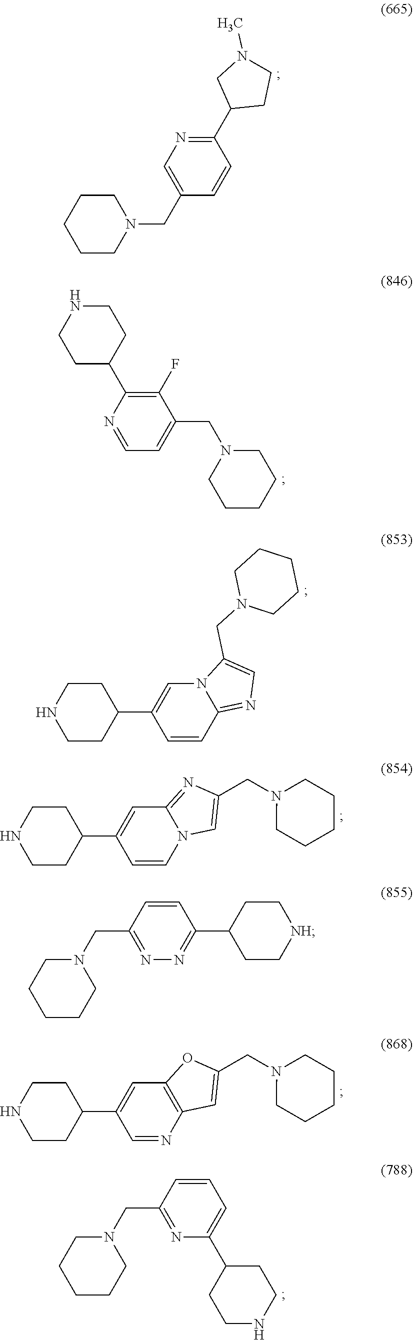 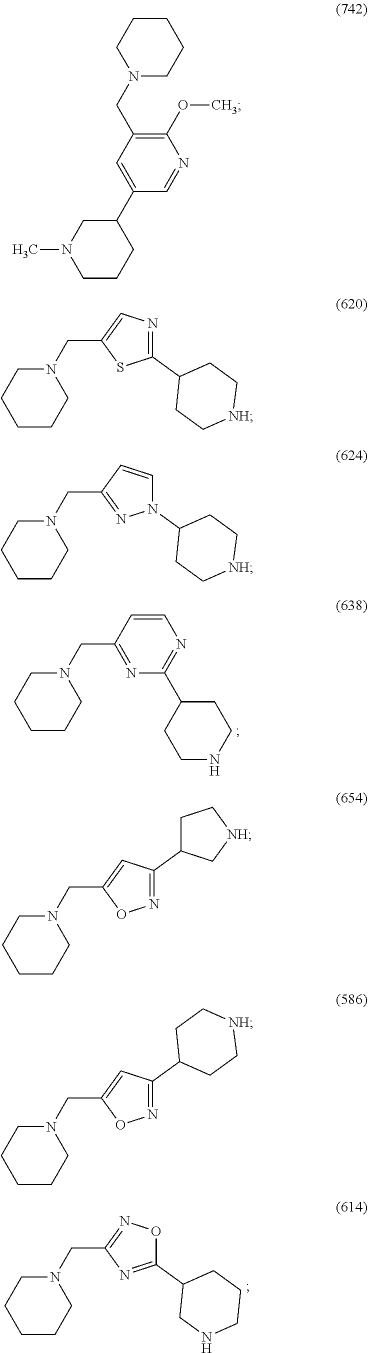 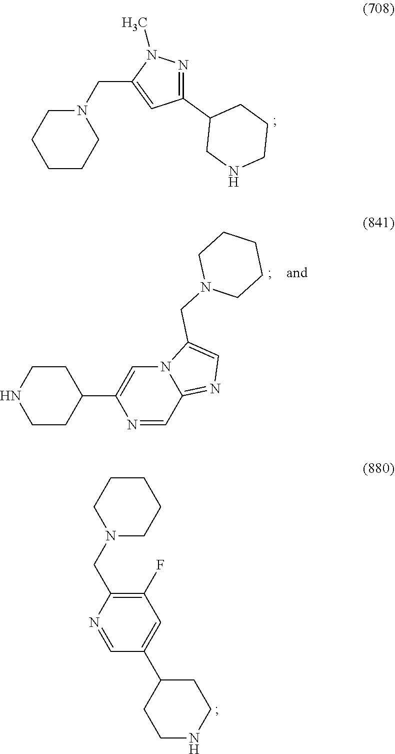 [SMILES: Cn1nc(C2CCCNC2)cc1CN1CCCCC1 ; c1nc2cnc(C3CCNCC3)cn2c1CN1CCCCC1 ; Fc1cc(C2CCNCC2)cnc1CN1CCCCC1]
and a pharmaceutically acceptable salt thereof.

14. The compound according to any one of the preceding claims, wherein the compound is a GBA inducer and increases glucocerebrosidase (GBA) enzyme levels and/or GBA enzyme activity.

15. A compound as defined in any one of claims 1-14 for use in the treatment of Parkinson's disease (PD).
